(19) United States
(12) Patent Application Publication
(10) Pub. No.: US 2025/0295791 A1
(43) Pub. Date: Sep. 25, 2025

(54) COLLAGEN-CONTAINING CURABLE FORMULATIONS

(71) Applicant: CollPlant Ltd., Rehovot (IL)

(72) Inventor: Roi MASHIACH, Ramle (IL)

(73) Assignee: CollPlant Ltd., Rehovot (IL)

(21) Appl. No.: 18/704,498

(22) PCT Filed: Oct. 27, 2022
(86) PCT No.: PCT/IL2022/051143

§ 371 Date: Apr. 25, 2024

Related U.S. Application Data
(60) Provisional application No. 63/272,313, filed on Oct. 27, 2021.

(51) Int. Cl. A61K 47/58; B33Y 10/00; B33Y 70/00

(52) U.S. Cl. CPC A61K 47/58; B33Y 10/00; B33Y 70/00

(57) ABSTRACT

A conjugate made of collagen and a plurality of curable elastic moieties covalently attached thereto, a curable formulation (e.g., a bioink composition) that comprises the conjugate and additive manufacturing of a three-dimensional object which utilizes the curable formulation are provided. Also provided are methods/processes of additive manufacturing that employ collagen that feature a plurality of photocurable groups, in which the viscosity of a collagen-containing formulation is determined by manipulating an amount of the photoinitiator that is mixed with the collagen.

### RELATED APPLICATION/S

This application claims the benefit of priority under 35 USC § 119 (e) of U.S. Provisional Patent Application No. 63/272,313 filed on Oct. 27, 2021, the contents of which are incorporated herein by reference in their entirety.

### SEQUENCE LISTING STATEMENT

The file entitled 94209.xml, created on Oct. 17, 2022, comprising 5,926,912 bytes, submitted concurrently with the filing of this application is incorporated herein by reference.

### FIELD AND BACKGROUND OF THE INVENTION

The present invention, in some embodiments thereof, relates to additive manufacturing and, more particularly, but not exclusively, to three-dimensional (3D) bioprinting of 3D objects using a collagen-based building material.

Collagen comprises the main component of connective tissue and is the most abundant protein in mammals, comprising approximately 30% of the proteins found in the body. Collagen serves as the predominant component and primary structural-mechanical determinant of most tissue extracellular matrix (ECM) [see, for example, Kadler K. Birth Defects Res C Embryo Today, 2004; 72:1-11; Kadler K E, Baldock C, Bella J, Boot-Handford R P. J Cell Sci. 2007; 120:1955-1958.; Kreger S T. Biopolymers. 2010 93 (8): 690-707].

Due to its unique characteristics and diverse profile in human body functions, collagen is frequently selected from a variety of biocompatible materials for use in tissue repair to support structural integrity, induce cellular infiltration and promote tissue regeneration. Among the 5 major collagen types, Type I collagen is the most abundant form of in the human body. Collagen's unique properties make it a favorite choice for regenerative medicine products.

Additive manufacturing (AM) is generally a process in which a three-dimensional (3D) object is manufactured utilizing a computer model of the objects. The basic operation of any AM system consists of slicing a three-dimensional computer model into thin cross sections, translating the result into two-dimensional position data and feeding the data to control equipment which manufacture a three-dimensional structure in a layerwise manner.

Various AM technologies exist, amongst which are stereolithography, digital light processing (DLP), and three-dimensional (3D) printing such as 3D inkjet printing. Such techniques are generally performed by layer-by-layer deposition and hardening (e.g., solidification) of one or more building materials, which typically include photopolymerizable (photocurable) materials.

Stereolithography, for example, is an additive manufacturing process which employs a liquid ultraviolet (UV)-curable building material and a UV laser. In such a process, for each dispensed layer of the building material, the laser beam traces a cross-section of the part pattern on the surface of the dispensed liquid building material. Exposure to the UV laser light cures and solidifies the pattern traced on the building material and joins it to the layer below. After being built, the formed parts are immersed in a chemical bath in order to be cleaned of excess building material and are subsequently cured in an UV oven,

In three-dimensional printing processes, for example, a building material is dispensed from a dispensing head having a set of nozzles or nozzle arrays to deposit layers on a receiving substrate. Depending on the building material, the layers may then be cured or solidify using a suitable device.

The building materials may include modeling material formulation(s) and support material formulation(s), which form, upon hardening, the object and the temporary support constructions supporting the object as it is being built, respectively. The modeling material formulation(s) is/are deposited to produce the desired object and the support material formulation(s) is/are used, with or without modeling material elements, to provide support structures for specific areas of the object during building and assure adequate vertical placement of subsequent object layers, e.g., in cases where objects include overhanging features or shapes such as curved geometries, negative angles, voids, and so on.

Both the modeling and support material formulations typically feature a viscosity that allows dispensing/depositing, and upon being dispensed and optionally exposed to curing/hardening, feature a higher viscosity. Both the modeling and support materials are preferably liquid at the working temperature at which they are dispensed, and are subsequently hardened, typically upon exposure to hardening or curing condition such as curing energy (e.g., UV curing), to form the required layer shape. After printing completion, support structures, if present, are removed to reveal the final shape of the fabricated 3D object. The hardening (curing) of the dispensed materials typically involves polymerization (e.g., photopolymerization) and/or crosslinking (e.g., photocrosslinking).

Additive manufacturing has been first used in biological applications for forming three-dimensional sacrificial resin molds in which 3D scaffolds from biological materials were created.

3D bioprinting is an additive manufacturing methodology which uses biological materials, optionally in combination with chemicals and/or cells, that are printed layer-by-layer with a precise positioning and a tight control of functional components placement to create a 3D structure,

Three dimensional (3D) bioprinting is gaining momentum in many medicinal applications, especially in regenerative medicine, to address the need for complex scaffolds, tissues and organs suitable for transplantation.

Inherent to 3D printing in general is that the mechanical properties of the printing media (the dispensed building material) are very different from the post-printed cured (hardened) material.

To allow tight control on the curing (e.g., polymerization) after printing, the building material commonly includes polymerizable (e.g., photopolymerizable) moieties or groups that polymerize (e.g., by chain elongation and/or cross-linking) upon being dispensed, to preserve the geometric shape and provide the necessary physical properties of the final product.

Different technologies have been developed for 3D bioprinting, including 3D Inkjet printing, Extrusion printing, Laser-assisted printing, digital light processing, and Projection stereolithography [see, for example, Murphy S V, Atala A, Nature Biotechnology. 2014 32 (8).; Miller J S, Burdick J. ACS Biomater. Sci. Eng. 2016, 2, 1658-1661]. Each technology has its different requirements for the dispensed building material (also referred to herein as printing media), which is derived from the specific application mechanism and the curing/gelation process required to maintain the 3D structure of the scaffold post printing.

For all technologies, the most important parameter determining the accuracy and efficiency of the printing is the static and dynamic physical properties of the dispensed building material, including viscosity, shear thinning and thixotropic properties. The static and dynamic properties of the building material are important not only for the printing technology but also when considering cell-laden printing, i.e. including cells in the building material dispensed during printing. In this case, the shearing forces applied to the building material during printing (dispensing) have a significant effect on the survival of the cells. Therefore, it is desirable to have good control on the specific properties of the printing media over a wide range of conditions, i.e. concentration, temperature, ionic strength and pH.

Type I collagen has been considered a perfect candidate for use as a major component of a building material in 3D-bioprinting.

Collagen methacrylate can be used as a rapidly self-assembling type I collagen to form cross-linked hydrogels for tissue engineering [see, for example, Isaacson et al., Experimental Eye Research 173, 188-193 (2018)]. It has been used with mesenchymal stem cells [Drzewiecki et al., A thermoreversible, photocrosslinkable collagen bio-ink for free-form fabrication of scaffolds for regenerative medicine, Technology (2017)], fibroblasts, adipose derived stem cells, epithelial cells, and many more. Collagen methacrylate is useful for forming scaffolds with varying degree of stiffness, by altering collagen concentration or the curing conditions (e.g., intensity and duration of irradiation).

Collagen (meth)acrylate extracted from tissues has been extensively characterized for its usefulness in 3D-bioprinting (extrusion, inkjet, and photolithographic [Drzewiecki, K. E. et al. Langmuir 30, 11204-11211 (2014); Gaudet, I. D. & Shreiber, D. I. Biointerphases 7, 25 (2012)].

Despite the significant advantages offered by this natural polymer, a number of factors hinder the use collagen (meth)acrylate 3D bioprinting. The use of tissue extracted collagen for this purpose is limited due to its sensitivity to temperature and ionic strength, which leads to spontaneous gel formation at temperatures higher than 20° C., under physiological conditions [see, for example, PureCol, Advanced BioMatrix, Inc.]. The typical temperature-dependent formation of gel of tissue extracted-collagens hampers significantly the precise fluidity during printing. Keeping the printing media at low temperature until application is a possible solution for this phenomena but implies a serious technical limitation. Another solution is the use of gelatin, the denatured form of collagen which does not become gel-like under these conditions. However, gelatin lacks the genuine tissue and cell interactions of native collagen and thus crucial biological functions are lost.

The present assignee has developed a technology that allows the purification of naïve human Type I collagen (rhCollagen) by introducing into tobacco plants, five human genes encoding heterotrimeric type I collagen [see, for example, Stein H. (2009) Biomacromolecules; 10:2640-5]. The protein is purified to homogeneity through a cost-effective industrial process taking advantage of collagen's unique properties. See also WO 2006/035442, WO 2009/053985, WO 2011/064773, WO 2013/093921, WO 2014/147622, and patents and patent applications deriving therefrom, all of which are incorporated by reference as if fully set forth herein.

WO 2018/225076, by the present assignee, describes formulations containing curable recombinant human collagen, and kits comprising same, which are usable in preparing, or as, modeling material formulations for additive manufacturing (e.g., 3D bioprinting) of 3D objects. The formulations feature a desired viscosity at a temperature higher than 10° C. (e.g., room temperature or 37° C.) and allow performing the additive manufacturing without cooling the system or a part thereof.

Additional background art includes U.S. Patent application Publication No. 2018/0193524; WO 2015/032985; Drzewiecki et al. (2014) Langmuir, 30 (37), 11204-11211; Ravichandran et al. (2015) Journal of Materials Chemistry B, 4 (2), 318-326; and Gaudet & Shreiber (2012) Biointerphases, 7 (1), 25,

Additional Background Art includes Zhang et al., Burns Trauma. 2022; 10: tkac010; WO 2022/093236; U.S. Patent Application Publication Nos. 2020/339925 and 2021/229364; U.S. Pat. No. 10,597,289; and CN 114958079.

### SUMMARY OF THE INVENTION

According to an aspect of some embodiments of the present invention there is provided a conjugate comprising collagen and a plurality of elastic/elastomeric moieties covalently attached to the collagen, wherein at least a portion of the elastic/elastomeric moieties feature a curable group.

According to some of any of the embodiments described herein, the curable group is at a terminus of each of the elastic/elastomeric moieties.

According to some of any of the embodiments described herein, the curable group is a photocurable or photopolymerizable group.

According to some of any of the embodiments described herein, the curable group is a (meth)acrylic group.

According to some of any of the embodiments described herein, at least a portion of the elastic/elastomeric moieties are poly(alkylene glycol)-containing moieties.

According to some of any of the embodiments described herein, at least a portion, or each, of the elastic/elastomeric moieties comprise a poly(alkylene glycol) moiety that features an acrylic or a (meth)acrylic group at its terminus.

According to some of any of the embodiments described herein, at least a portion of the elastic/elastomeric moieties are covalently attached to lysine residues of the collagen.

According to some of any of the embodiments described herein, at least 1%, for example, from 1 to 20, or from 1 to 10%, of lysine residues in the collagen have the elastic/elastomeric moieties covalently attached thereto.

According to some of any of the embodiments described herein, at least a portion of the elastic/elastomeric moieties is attached to the lysine residues via a carbamate bond.

According to some of any of the embodiments described herein, the collagen features a plurality of curable groups, e.g., photocurable groups (in addition to the curable elastic/elastomeric moieties).

According to some of any of the embodiments described herein, the collagen is a human Type I collagen.

According to some of any of the embodiments described herein, the collagen is a recombinant collagen.

According to some of any of the embodiments described herein, the collagen is a plant-derived recombinant collagen.

According to some of any of the embodiments described herein, the collagen is a plant-derived recombinant human Type I collagen.

According to an aspect of some embodiments of the present invention there is provided a curable formulation comprising the conjugate as described herein in any of the respective embodiments and any combination thereof.

According to some of any of the embodiments described herein, the curable formulation further comprises an aqueous carrier.

According to some of any of the embodiments described herein, a concentration of the conjugate ranges from 0.5 mg/mL to 50 mg/mL, or from 0.5 mg/mL to 20 mg/mL, or from 0.5 mg/mL to 10 mg/mL, or from 1 mg/mL to 10 mg/mL.

According to some of any of the embodiments described herein, the curable formulation further comprises at least one additional curable material.

According to some of any of the embodiments described herein, the additional material features curable (e.g., photocurable) groups.

According to some of any of the embodiments described herein, the additional material is or comprises a poly(alkylene glycol) that features at least one (meth)acrylic group at its terminus.

According to some of any of the embodiments described herein, a concentration of the additional curable material ranges from 1 to 20, or from 1 to 10, % by weight of the total weight of the formulation.

According to some of any of the embodiments described herein, the curable formulation further comprises a biological material other than the curable collagen.

According to some of any of the embodiments described herein, the curable formulation further comprises an agent that promotes polymerization of the conjugate.

According to some of any of the embodiments described herein, the curable group is a photocurable group and the agent is a photoinitiator.

According to some of any of the embodiments described herein, the curable formulation further comprises a dye substance that is capable of absorbing light at a wavelength of from 300 nm to 800 nm, or of from 300 to 450 nm.

According to some of any of the embodiments described herein, the dye substance has a plurality of negatively-charged groups.

According to some of any of the embodiments described herein, the dye substance is Vitamin B12.

According to some of any of the embodiments described herein, the dye substance is minocycline.

According to some of any of the embodiments described herein, the dye substance is a quinoline.

According to some of any of the embodiments described herein, an amount of the dye substance ranges from 0.01 to 5% by weight of the total weight of the composition, as described herein in any of the respective embodiments and any combination thereof.

According to an aspect of some embodiments of the present invention there is provided a process of additive manufacturing a three-dimensional object featuring, in at least a portion thereof, a collagen-based material, the process comprising dispensing at least one modeling material formulation to sequentially form a plurality of layers in a configured pattern corresponding to a shape of the object, wherein for at least a portion of the layers, the dispensing is of a modeling material formulation that comprises the curable formulation as described herein in any of the respective embodiments and any combination thereof, thereby manufacturing the three-dimensional object.

According to some of any of the embodiments described herein, the process further comprises exposing at least the portion of the layers to a curing condition suitable for hardening the curable formulation.

According to some of any of the embodiments described herein, for at least a portion of the layers, the dispensing is further of a modeling material formulation that comprises an agent that modifies a mechanical and/or rheological and/or physical property of the formulation and/or of a respective portion of the object.

According to some of any of the embodiments described herein, for at least a portion of the layers, the dispensing is further of a modeling material formulation that comprises a biological material other than the human recombinant collagen.

According to an aspect of some embodiments of the present invention there is provided a three-dimensional biological object obtainable by the process as described herein in any of the respective embodiments and any combination thereof.

According to some of any of the embodiments described herein, the object further comprises a biological material other than the collagen-based material in or on at least a portion thereof.

According to some of any of the embodiments described herein, the three-dimensional biological object is for use in repairing a damaged tissue.

According to some of any of the embodiments described herein, the three-dimensional biological object is for use as an artificial tissue or organ.

According to an aspect of some embodiments of the present invention there is provided a curable formulation for use in additive manufacturing of a three-dimensional object, the formulation comprising a photocurable biological material and a dye substance capable of absorbing light at a wavelength of from 300 nm to 800 nm, the dye substance comprising Vitamin B12.

According to some of any of the embodiments described herein, an amount of the dye substance ranges from 0.01 to 5% by weight of the total weight of the composition.

According to some of any of the embodiments described herein, the photocurable biological material comprises a collagen that features a plurality of photocurable groups.

According to some of any of the embodiments described herein, the photocurable groups comprise (meth)acrylic groups,

According to some of any of the embodiments described herein, the photocurable groups are attached to the collagen directly.

According to some of any of the embodiments described herein, the collagen comprises a plurality of elastic/elastomeric moieties covalently attached thereto, at least a portion of the elastic moieties featuring the photocurable group.

According to some of any of the embodiments described herein, the collagen is a human Type I collagen, e.g., a recombinant human collagen, for example, a plant-derived recombinant human Type I collagen.

According to an aspect of some embodiments of the present invention there is provided a process of additive manufacturing a three-dimensional object featuring, in at least a portion thereof, a biological material, the process comprising dispensing at least one modeling material formulation to sequentially form a plurality of layers in a configured pattern corresponding to a shape of the object, wherein for at least a portion of the layers, the dispensing is of a modeling material formulation that comprises the vitamin B12-containing curable formulation as described herein in any of the respective embodiments and any combination thereof, thereby manufacturing the three-dimensional object,

According to some of any of the embodiments described herein, the process further comprises exposing the portion of the layers to irradiation suitable for hardening the formulation.

According to an aspect of some embodiments of the present invention there is provided a process of additive manufacturing a three-dimensional object featuring, in at least a portion thereof, a collagen-based material, the process comprising:
selecting an additive manufacturing technique;
mixing at least a collagen that features a plurality of photocurable groups, a photoinitiator, optionally an aqueous carrier, and optionally other curable and/or non-curable components, to thereby prepare a modeling material formulation (e.g., a bioink composition), wherein an amount of the photoinitiator is selected so as to provide a viscosity that is suitable for dispensing the formulation in the additive manufacturing technique;
dispensing at least one modeling material formulation to sequentially form a plurality of layers in a configured pattern corresponding to a shape of the object,
wherein for at least a portion of the layers, the dispensing is of the modeling material formulation that comprises the collagen that features a plurality of photocurable groups,
thereby manufacturing the three-dimensional object.

According to some of any of the embodiments described herein, the collagen is a human Type I collagen, as described herein in any of the respective embodiments and any combination thereof.

According to some of any of the embodiments described herein, the photocurable groups comprise (meth)acrylic groups.

According to some of any of the embodiments described herein, the photoinitiator is an acyl phosphine oxide type photoinitiator.

According to some of any of the embodiments described herein, the photoinitiator is phenyl-2,4,6-trimethylbenzoylphosphine oxide or a salt thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic presentation of (meth)acrylated PEG moieties conjugated to lysine residues of collagen (recombinant human Type I collagen), according to some embodiments of the present invention.

FIG. 2 presents SDS-PAGE comparing collagen and a collagen-methacrylated PEG conjugate (CPM), and reference mixture of proteins with known molecular weights (“ladder”) as indicated,

FIGS. 3A-B present plots showing the viscosity of a formulation containing a collagen-methacrylated PEG conjugate (CPM; FIG. 3A), and of a formulation containing methacrylated collagen (CMR; FIG. 3B), as a function of shear rate and upon 1, 3, or 5 minutes relaxation.

FIGS. 4A-B present the recovery of a viscosity of a formulation containing a collagen-methacrylated PEG conjugate (CPM; FIG. 4A), and of a formulation containing methacrylated collagen (CMR; FIG. 4B), upon manipulating the shear force, presenting the recovery of the tested formulations.

FIG. 5 presents comparative plots showing the storage modulus (G′) of a formulation comprising methacrylated rh-Collagen (CMR), a formulation containing an exemplary methacrylated Collagen-PEG conjugate according to some of the present embodiments, (CPM) a formulation devoid of a protein.

FIG. 6 presents photographs of a solution of methacrylated collagen (left vial) and a solution an exemplary methacrylated Collagen-PEG conjugate according to some of the present embodiments (right vial), upon addition of a polysufate dye.

FIG. 7 presents comparative plots showing the viscosity of a CMR formulation containing vitamin B12 or 4-nitrophenol as a dye at various sheer rates.

FIG. 8 presents comparative plots showing the effect of the amount of the photoinitiator (LAP) on the viscosity of formulations containing CMR.

FIG. 9 presents comparative plots showing the storage modulus (G′) of formulations comprising methacrylated rh-Collagen (CMR; 5 mg/mL), LAP (0.5% by weight, and varying concentrations of minocycline.

FIG. 10 presents a schematic illustration of the synthesis of a collagen (recombinant human Type I collagen) having a plurality of (meth)acrylate group and a plurality of PEG moieties conjugated to lysine residues thereof (lower scheme) and of an intermediate reagent used for its preparation (upper scheme).

### DESCRIPTION OF SPECIFIC EMBODIMENTS OF THE INVENTION

The present invention, in some embodiments thereof, relates to additive manufacturing and, more particularly, but not exclusively, to three-dimensional (3D) bioprinting of 3D objects using a collagen-based building material.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

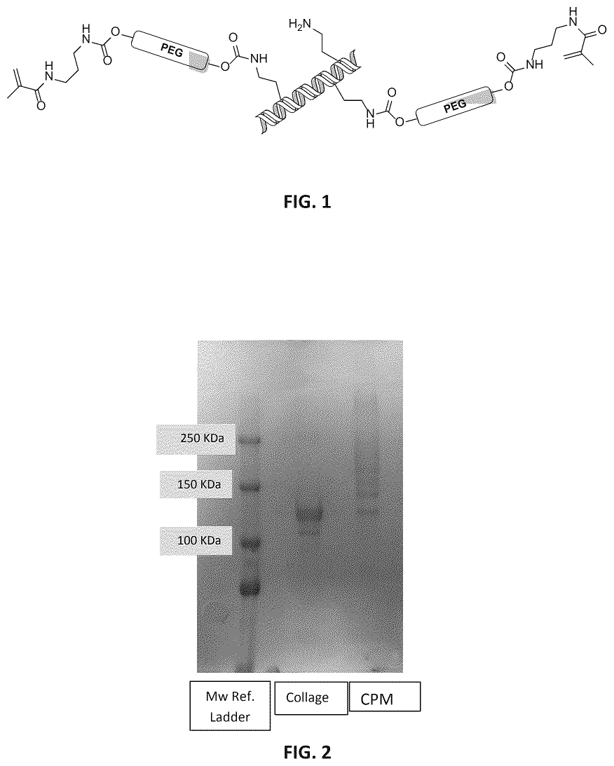

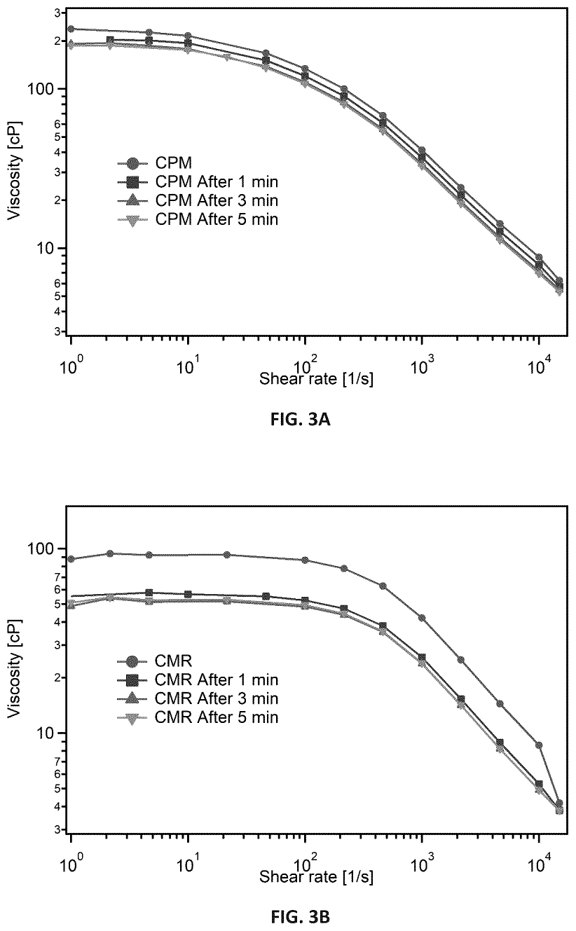

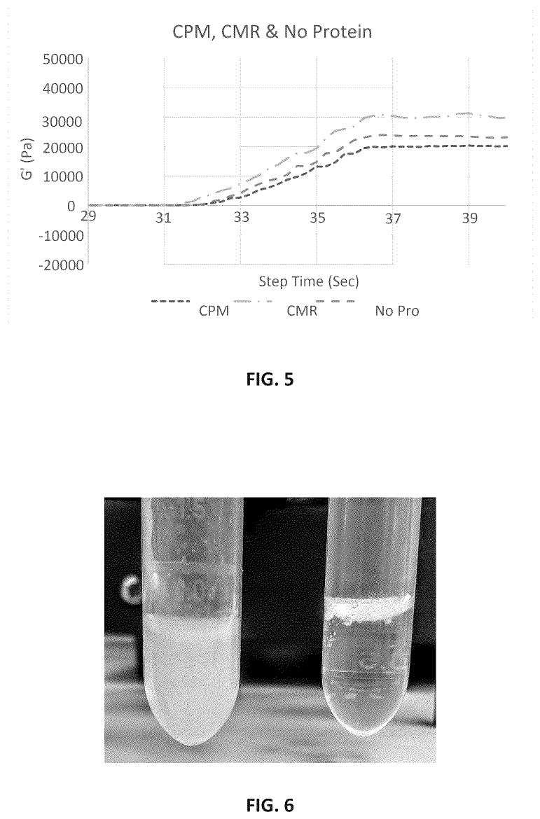

In a search for a curable collagen material that exhibits improved mechanical properties, and can be beneficially utilized in bioink compositions usable for additive manufacturing of three-dimension objects that comprise collagen, the present inventors have devised and successfully prepared and practiced a conjugate that comprises collagen having attached thereto curable elastic/elastomeric moieties. An exemplary such a conjugate is schematically illustrated in FIG. 1. The present inventors have demonstrated that formulations containing such a conjugate exhibit improved recovery under shear and improved modulus, as shown in FIGS. 3A-B and 4A-B, improved properties of the hardened material, as shown in FIG. 5, and further, allow using absorbing dye additives that feature negatively charged groups without adversely affecting the formulation consistency, as exemplified in FIG. 6.

Embodiments of the present invention relate to conjugates of collagen and curable elastic moieties, to bioink compositions comprising such conjugates and to uses thereof in additive manufacturing,

In the course of studying bioink compositions that comprise curable collagen, the present inventors have further uncovered that vitamin B12 can be advantageously used as an absorbing dye substance in such bioink compositions, as is demonstrated in FIG. 7.

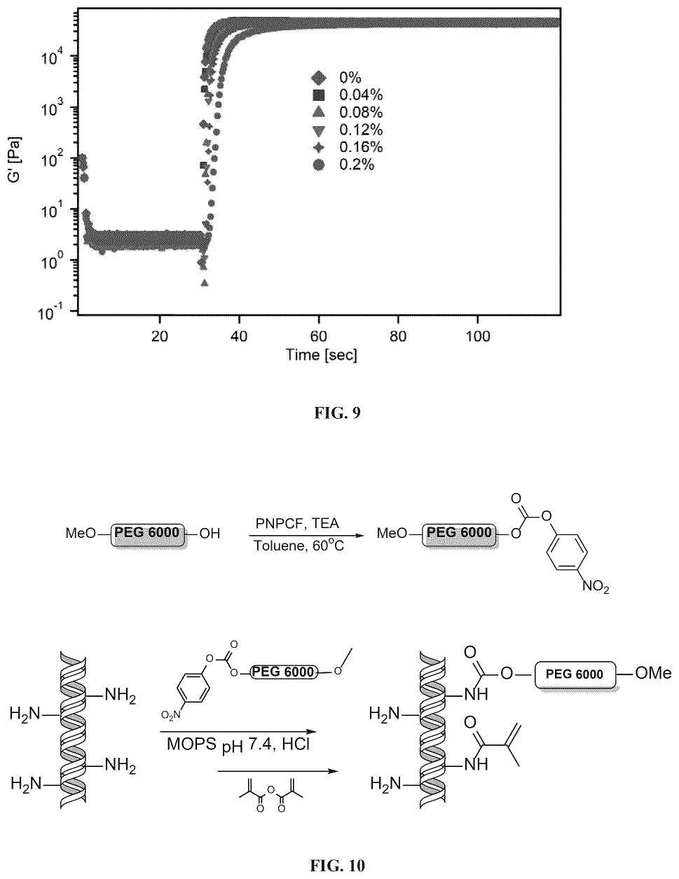

In the course of studying bioink compositions that comprise curable collagen, the present inventors have further uncovered that minocycline can be advantageously used as an absorbing dye substance in such bioink compositions, as is demonstrated in FIG. 9.

Embodiments of the present invention therefore further relate to curable formulations (e.g., collagen-based bioink compositions as described herein) that comprise vitamin B12 as an absorbing dye substance, as described herein.

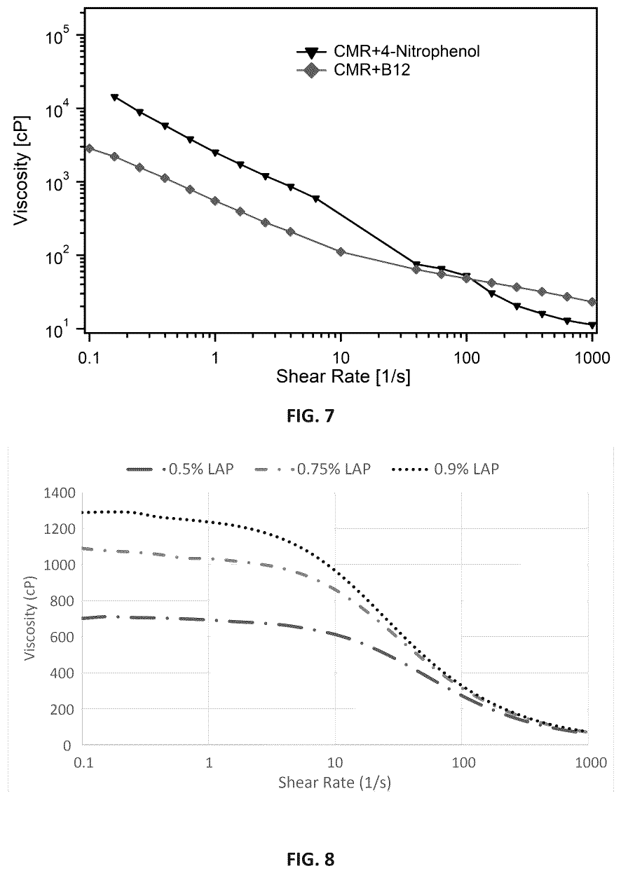

The present inventors have further uncovered that a curable collagen as described herein interacts with a common type of photoinitiators (acylphosphine oxide type photoinitiators such as Lithium phenyl-2,4,6-trimethylbenzoylphosphinate (LAP)), such that the amount of the photoinitiator affects the viscosity of the curable formulation containing same (before exposure to curing irradiation), as exemplified in FIG. 8.

Embodiments of the present invention therefore further relate to curable formulations that comprise variable amounts of a photoinitiator, and which can be designed and/or selected accordingly so as to suit a respective additive manufacturing methodology, as described herein.

### Collagen:

The conjugate according to the present embodiments comprises a collagen to which are attached curable elastic moieties.

The term “collagen” as used herein, refers to a polypeptide having a triple helix structure and containing a repeating Gly-X-Y triplet, where X and Y can be any amino acid but are frequently the imino acids proline and hydroxyproline. According to one embodiment, the collagen is a type I, II, III, V, XI, or biologically active fragments therefrom.

A collagen according to some of the present embodiments also refers to homologs (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to collagen sequences such as listed in Table A as determined using BlastP software of the National Center of Biotechnology Information (NCBI) using default parameters). The homolog may also refer to a deletion, insertion, or substitution variant, including an amino acid substitution, thereof and biologically active polypeptide fragments thereof.

According to a particular embodiment, the collagen is a human collagen.

In another embodiment, the collagen comprises a naturally occurring amino acid sequence of human collagen.

Table A below lists examples of collagen NCBI sequence numbers.

TABLE A

| Exemplary procollagen NCBI sequence | |
|---|---|
| number | SEQ ID NO: |
| P02452 | 1 |
| P08123 | 2 |

The annotation of SEQ ID NO: 1 is as follows:
Amino acids 1-22—signal peptide;
Amino acids 23-161—N-terminal peptide;
Amino acids 162-1218—collagen alpha-1 (I) chain;
Amino acids 1219-1464—C-terminal peptide; The annotation of SEQ ID NO: 2 is as follows:
Amino acids 1-22—signal peptide;
Amino acids 23-79—N-terminal peptide;
Amino acids 80-1119—collagen alpha-2 (I) chain;
Amino acids 1120-1366—C-terminal peptide.

According to one embodiment, the collagen comprises a sufficient portion of its telopeptides such that under suitable conditions it is capable of forming fibrils.

Thus, for example, the collagen may be atelocollagen, a telocollagen or procollagen,

As used herein, the term “atelocollagen” refers to collagen molecules lacking both the N- and C-terminal propeptides typically comprised in procollagen and at least a portion of its telopeptides, but including a sufficient portion of its telopeptides such that under suitable conditions it is capable of forming fibrils.

The term “procollagen” as used herein, refers to a collagen molecule (e.g. human) that comprises either an N-terminal propeptide, a C-terminal propeptide or both. Exemplary human procollagen amino acid sequences are set forth by SEQ ID NOs: 3, 4, 5 and 6.

The term “telocollagen” as used herein, refers to collagen molecules that lack both the N- and C-terminal propeptides typically comprised in procollagen but still contain the telopeptides. The telopeptides of fibrillar collagen are the remnants of the N- and C-terminal propeptides following digestion with native N/C proteinases.

According to another embodiment, the collagen is devoid of its telopeptides and is not capable of undergoing fibrillogenesis.

According to another embodiment, the collagen is a mixture of the types of collagen above.

According to a particular embodiment, the collagen is genetically engineered using recombinant DNA technology (e.g., human collagen).

Methods of isolating collagen from animals are known in the art. Dispersal and solubilization of native animal collagen can be achieved using various proteolytic enzymes (such as porcine mucosal pepsin, bromelain, chymopapain, chymotrypsin, collagenase, ficin, papain, peptidase, proteinase A, proteinase K, trypsin, microbial proteases, and, similar enzymes or combinations of such enzymes) which disrupt the intermolecular bonds and remove the immunogenic non-helical telopeptides without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). The resulting soluble collagen can be subsequently purified by repeated precipitation at low pH and high ionic strength, followed by washing and re-solublization at low pH.

Plants expressing collagen chains and procollagen are known in the art, see for example, WO 06/035442; Merle et al., FEBS Lett. 2002 Mar. 27; 515(1-3):114-8. PMID: 11943205; and Ruggiero et al., 2000, FEBS Lett. 2000 Mar. 3; 469(1):132-6. PMID: 10708770; and U.S. Patent Applications Publication Nos. 2002/098578 and 2002/0142391, as well as U.S. Pat. No. 6,617,431, each of which are incorporated herein by reference.

It will be appreciated that embodiments of the present invention also contemplate genetically modified forms of collagen/atelocollagen—for example collagenase-resistant collagens and the like [see, for example, Wu et al., Proc Natl. Acad Sci, Vol. 87, p. 5888-5892, 1990].

Recombinant procollagen or telocollagen (e.g. human) may be expressed in any non-animal cell, including but not limited to plant cells and other eukaryotic cells such as yeast and fungus.

Plants in which procollagen or telocollagen may be produced (i.e. expressed) may be of lower (e.g. moss and algae) or higher (vascular) plant species, including tissues or isolated cells and extracts thereof (e.g. cell suspensions). Preferred plants are those which are capable of accumulating large amounts of collagen chains, collagen and/or the processing enzymes described herein below. Such plants may also be selected according to their resistance to stress conditions and the case at which expressed components or assembled collagen can be extracted. Examples of plants in which human procollagen may be expressed include, but are not limited to tobacco, maize, alfalfa, rice, potato, soybean, tomato, wheat, barley, canola, carrot, lettuce and cotton,

Production of recombinant procollagen is typically effected by stable or transient transformation with an exogenous polynucleotide sequence encoding human procollagen.

Exemplary polynucleotide sequences encoding human procollagen are set forth by SEQ ID NOs: 7, 8, 9 and 10.

Production of human telocollagen is typically effected by stable or transient transformation with an exogenous polynucleotide sequence encoding human procollagen and at least one exogenous polynucleotide sequence encoding the relevant protease. Alternatively, a protease may be added following isolation of the recombinant procollagen.

The stability of the triple-helical structure of collagen requires the hydroxylation of prolines by the enzyme prolyl-4-hydroxylase (P4H) to form residues of hydroxyproline within the collagen chain. Although plants are capable of synthesizing hydroxyproline-containing proteins, the prolyl hydroxylase that is responsible for synthesis of hydroxyproline in plant cells exhibits relatively loose substrate sequence specificity as compared with mammalian P4H. Thus, production of collagen containing hydroxyproline only in the Y position of Gly-X-Y triplets requires co-expression of collagen and human or mammalian P4H genes [Olsen et al, Adv Drug Deliv Rev. 2003 Nov. 28; 55(12):1547-67],

Thus, according to one embodiment, the procollagen or telocollagen is expressed in a subcellular compartment of a plant that is devoid of endogenous P4H activity.

As used herein, the phrase “subcellular compartment devoid of endogenous P4H activity” refers to any compartmentalized region of the cell which does not include plant P4H or an enzyme having plant-like P4H activity. According to one embodiment, the subcellular compartment is a vacuole, an apoplast or a chloroplast. According to a particular embodiment, the subcellular compartment is a vacuole.

According to another embodiment, the subcellular compartment is an apoplast.

Accumulation of the expressed procollagen in a subcellular compartment devoid of endogenous P4H activity can be effected via any one of several approaches.

For example, the expressed procollagen/telocollagen can include a signal sequence for targeting the expressed protein to a subcellular compartment such as the apoplast or an organelle (e.g. chloroplast).

Examples of suitable signal sequences include the chloroplast transit peptide (included in Swiss-Prot entry P07689, amino acids 1-57) and the Mitochondrion transit peptide (included in Swiss-Prot entry P46643, amino acids 1-28). Targeting to the vacuole may be achieved by fusing the polynucleotide sequence encoding the collagen to a vacuolar targeting sequence—for example using the vacuolar targeting sequence of the thiol protease aleurain precursor (NCBI accession P05167 GI: 113603):-MAHARVLLLALAVLATAAVAVASSSSFADSNPIRPVTDRAASTLA (SEQ ID NO: 14). Typically, the polynucleotide sequence encoding the collagen also comprises an ER targeting sequence. In one embodiment, the ER targeting sequence is native to the collagen sequence. In another embodiment, the native ER targeting sequence is removed and a non-native ER targeting sequence is added. The non-native ER targeting sequence may be comprised in the vacuolar targeting sequence. It will be appreciated, for it to traverse the ER and move on to the vacuole, the collagen sequence should be devoid of an ER retention sequence.

Alternatively, the sequence of the procollagen can be modified in a way which alters the cellular localization of the procollagen when expressed in plants.

The present invention contemplates genetically modified cells co-expressing both human procollagen and a P4H. In one embodiment, the P4H is capable of correctly hydroxylating the procollagen alpha chain(s) [i.e. hydroxylating only the proline (Y) position of the Gly-X-Y triplets]. P4H is an enzyme composed of two subunits, alpha and beta as set forth in Genbank Nos. P07237 and P13674. Both subunits are necessary to form an active enzyme, while the beta subunit also possesses a chaperon function.

The P4H expressed by the genetically modified cells of the present invention is preferably a human P4H. An exemplary polynucleotide sequence which encodes human P4H is SEQ ID Nos; 11 and 12. In addition, P4H mutants which exhibit enhanced substrate specificity, or P4H homologues can also be used. A suitable P4H homologue is exemplified by an Arabidopsis oxidoreductase identified by NCBI accession no: NP_179363.

Since it is essential that P4H co-accumulates with the expressed procollagen chain, the coding sequence thereof is preferably modified accordingly (e.g. by addition or deletion of signal sequences). Thus, the present invention contemplates using P4H polynucleotide sequences that are fused to vacuole targeting sequences. It will be appreciated that for targeting to the vacuole, when an endogenous ER retention sequence is present, it should be removed prior to expression.

In mammalian cells, collagen is also modified by Lysyl hydroxylase, galactosyltransferase and glucosyltransferase. These enzymes sequentially modify lysyl residues in specific positions to hydroxylysyl, galactosylhydroxylysyl and glucosylgalactosyl hydroxylysyl residues at specific positions. A single human enzyme, Lysyl hydroxylase 3 (LH3), as set forth in Genbank No. O60568, can catalyze all three consecutive modifying steps as seen in hydroxylysine-linked carbohydrate formation,

Thus, genetically modified cells according to some embodiments may also express mammalian LH3 (optionally fused to vacuole targeting sequences). It will be appreciated that for targeting to the vacuole, the endogenous ER retention sequence is removed prior to expression.

An LH3 encoding sequence such as that set forth by SEQ ID NO: 13, can be used for such purposes.

The procollagen(s) and modifying enzymes described above can be expressed from a stably integrated or a transiently expressed nucleic acid construct which includes polynucleotide sequences encoding the procollagen alpha chains and/or modifying enzymes (e.g. P4H and LH3) positioned under the transcriptional control of functional promoters. Such a nucleic acid construct (which is also termed herein as an expression construct) can be configured for expression throughout the whole organism (e.g. plant, defined tissues or defined cells), and/or at defined developmental stages of the organism. Such a construct may also include selection markers (e.g. antibiotic resistance), enhancer elements and an origin of replication for bacterial replication.

There are various methods for introducing nucleic acid constructs into both monocotyledonous and dicotyledenous plants (Potrykus, I., Annu. Rev. Plant, Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct, in which case these sequences are not inherited by the plant's progeny.

In addition, several methods exist in which a nucleic acid construct can be directly introduced into the DNA of a DNA-containing organelle such as a chloroplast.

There are two principle methods of effecting stable genomic integration of exogenous sequences, such as those included within the nucleic acid constructs of the present invention, into plant genomes:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Regardless of the transformation technique employed, once collagen-expressing progeny are identified, such plants are further cultivated under conditions which maximize expression thereof. Progeny resulting from transformed plants can be selected, by verifying presence of exogenous mRNA and/or polypeptides by using nucleic acid or protein probes (e.g. antibodies), The latter approach enables localization of the expressed polypeptide components (by for example, probing fractionated plants extracts) and thus also verifies the plant's potential for correct processing and assembly of the foreign protein.

Following cultivation of such plants, the telopeptide-comprising collagen is typically harvested, Plant tissues/cells are preferably harvested at maturity, and the procollagen molecules are isolated using extraction approaches. Preferably, the harvesting is effected such that the procollagen remains in a state that it can be cleaved by protease enzymes. According to one embodiment, a crude extract is generated from the transgenic plants of the present invention and subsequently contacted with the protease enzymes.

As mentioned, the propeptide or telopeptide-comprising collagen may be incubated with a protease to generate atelocollagen or collagen prior to solubilization. It will be appreciated that the propeptide or telopeptide-comprising collagen may be purified from the genetically engineered cells prior to incubation with protease, or alternatively may be purified following incubation with the protease. Still alternatively, the propeptide or telopeptide-comprising collagen may be partially purified prior to protease treatment and then fully purified following protease treatment, Yet alternatively, the propeptide or telopeptide-comprising collagen may be treated with protease concomitant with other extraction/purification procedures.

Exemplary methods of purifying or semi-purifying the telopeptide-comprising collagen of the present invention include, but are not limited to salting out with ammonium sulfate or the like and/or removal of small molecules by ultrafiltration.

The protease used for cleaving the recombinant propeptide or telopeptide comprising collagen is not necessarily derived from an animal. Exemplary proteases include, but are not limited to certain plant derived proteases e.g. ficin (EC 3.4.22.3) and certain bacterial derived proteases e.g. subtilisin (EC 3.4.21.62), neutrase. The present inventors also contemplate the use of recombinant enzymes such as rhTrypsin and rhPepsin. Several such enzymes are commercially available e.g. Ficin from Fig tree latex (Sigma, catalog #F4125 and Europe Biochem), Subtilisin from Bacillus licheniformis (Sigma, catalog #P5459) Neutrase from bacterium Bacillus amyloliquefaciens (Novozymes, catalog #PW201041) and TrypZcan™, a recombinant human trypsin expressed in corn (Sigma catalog #T3449).

In some of any of the embodiments described herein, the recombinant human collagen is a recombinant human type I collagen.

In some of any of the embodiments described herein, the recombinant human collagen is a plant-derived recombinant human collagen and in some embodiments the plant is tobacco. An exemplary collagen is described in Stein H. (2009) Biomacromolecules; 10:2640-5, WO 2006/035442, WO 2009/053985, WO 2011/064773, WO 2013/093921, and WO 2014/147622.

In some of any of the embodiments described herein, the recombinant human collagen is a recombinant human type I collagen comprising two al units having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:15 as determined using BlastP software of the National Center of Biotechnology Information (NCBI) using default parameters), and one α2 unit having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:6. According to a particular embodiment, the type I collagen consists of two al units which consists of the sequence as set forth in SEQ ID NO: 15 and one α2 unit consisting of the sequence as set forth in SEQ ID NO:6, as determined using BlastP software of the National Center of Biotechnology Information (NCBI) using default parameters).

In some of any of the embodiments described herein, the al unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 16. The α2 unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 10.

Identity (e.g., percent homology) can be determined using any homology comparison software, including for example, the BlastN software of the National Center of Biotechnology Information (NCBI) such as by using default parameters.

In some of any of the embodiments described herein, the human recombinant collagen (rhCollagen) as described herein in any of the respective embodiments is a monomeric rhCollagen.

By “monomeric” it is meant a rhCollagen as described herein which is soluble in an aqueous solution and does not form fibrillar aggregates.

In some of any of the embodiments described herein, the human recombinant collagen (rhCollagen) as described herein in any of the respective embodiments is a fibrillar rhCollagen.

By “fibrillar” it is meant a rhCollagen as described herein which is in a form of fibrillar aggregates in an aqueous solution containing same. Typically, but not obligatory, fibrillar rhCollagen is formed by subjecting monomeric rhCollagen to a fibrillogenesis buffer, typically featuring a basic pH. An exemplary procedure for forming fibrillar rhCollagen, is described in WO 2018/225076.

### Curable Collagen:

Some embodiments of the present invention relate to curable collagen.

By “curable” it is meant herein a material that is capable of undergoing curing, or hardening (e.g., a change in viscosity or in G′), as defined herein, when exposed to a suitable curing condition.

A curable material is typically hardened or cured by undergoing polymerization and/or cross-linking.

Curable materials are typically polymerizable materials, which undergo polymerization and/or cross-linking when exposed to a suitable curing condition or a suitable curing energy (a suitable energy source). Alternatively, curable materials are thermo-responsive materials, which solidify or harden upon exposure to a temperature change (e.g., heating or cooling). Optionally, curable materials are made of small particles (e.g., nanoparticles or nanoclays) which can undergo curing to form a hardened material. Further optionally, curable materials are biological materials which undergo a reaction to form a hardened or solid material upon a biological reaction (e.g., an enzymatically-catalyzed reaction).

In some of any of the embodiments described herein, a curable material is a photopolymerizable material, which polymerizes and/or undergoes cross-linking upon exposure to radiation, as described herein, and in some embodiments the curable material is a UV-curable material, which polymerizes or undergoes cross-linking upon exposure to UV-vis radiation, as described herein.

In some of any of the embodiments described herein, when a curable material is exposed to a curing condition (e.g., radiation, reagent), it polymerizes by any one, or combination, of chain elongation, entanglement and cross-linking. The cross-linking can be chemical and/or physical.

In some of any of the embodiments described herein, a curable material can be a mono-functional curable material or a multi-functional curable material.

Herein, a mono-functional curable material comprises one curable group-a functional group that can undergo polymerization, entanglement and/or cross-linking when exposed to a curing condition (e.g., radiation, presence of calcium ions).

A multi-functional curable material comprises two or more, e.g., 2, 3, 4 or more, curable groups. Multi-functional curable materials can be, for example, di-functional, tri-functional or tetra-functional curable materials, which comprise 2, 3 or 4 curable groups, respectively.

By “curable collagen” it is meant a collagen as described herein in any of the respective embodiments (human recombinant collagen), which features one or more curable groups as defined herein. According to some of any of the embodiments described herein, the curable collagen is a multi-functional curable material that comprises a plurality of curable groups, as defined herein.

The terms “curable collagen”, “curable rhCollagen”, “collagen featuring one or more (or at least one) curable groups” and “rhCollagen featuring one or more (or at least one) curable groups” are used herein interchangeably.

According to some of any of the embodiments described herein, the curable collagen comprises an amino acid sequence as described herein in any of the respective embodiments, and features one or more, preferably a plurality of, curable groups generated at at least a portion of the amino acid residues forming the collagen, preferably by covalent attachment of a compound that comprises a curable group to functional groups of the side chains of the amino acid residues. Alternatively, or in addition, curable groups can be generated at the N-terminus and/or C-terminus of one or more the units forming the collagen, for example, by covalent attachment of a compound that comprises a curable group to a respective amine or carboxylate.

According to some of any of the embodiments described herein, the curable collagen is as described in WO 2018/225076.

According to some of any of the embodiments described herein, a curable collagen describes a collagen as described herein (e.g., rhCollagen as described herein in any of the respective embodiments) to which one or more curable groups are attached directly (e.g., by means of a covalent bond to a respective lysine residue of the collagen), or are not attached by means of an elastic moiety that terminates by a curable group as described herein. It is to be noted however that a curable collagen can further comprise one or more elastic moieties that terminate by a curable group as described herein in the context of a conjugate.

According to some of any of the embodiments described herein, at least a portion of the curable groups in a curable collagen as described herein are cross-linkable groups, which undergo cross-linking when exposed to a curing condition.

In some embodiments, curable groups can undergo polymerization and/or cross-linking via free-radical mechanism.

Exemplary such curable groups include acrylic groups, including acrylate, methacrylate, acrylamide and methacrylamide groups, which are collectively referred to herein as (meth)acrylic groups. Other free-radical curable groups may include thiols, vinyl ethers and other groups that feature a reactive double bond.

In some embodiments, curable groups can undergo polymerization and/or cross-linking via other mechanisms, such as cationic polymerization, or (cationic or anionic) ring opening polymerization. Exemplary such curable groups include, but are not limited to, epoxy-containing groups, caprolactam, caprolactone, oxetane, and vinyl ether.

Other curable groups can include, for example, formation of amide bonds between functional carboxylate and amine group (each being a curable group that reacts with the other and can effect cross-linking); formation of an imine bond between and amine and an aldehyde group; formation of urethane between isocyanate groups and hydroxyl groups via polycondensation in the presence of a catalyst and/or upon exposure to UV radiation; and formation of disulfide bonds between two thiols.

Any other curable groups are contemplated.

The curable groups in the curable collagen can be generated by means of chemical reactions between a material that comprises or can generate the curable group(s) when reacted with chemically-compatible functional groups present in the collagen, as described herein, either directly, or be means of a spacer or a linker, using chemistries well known in the art. For example, a material that comprises a curable group and a functional group can be reacted with a compatible functional group in the collagen, for example, a functional group in an amino acid side chain, such that the curable group is a substituent of the amino acid side chain.

In some embodiments, a compatible functional group is first generated within the collagen by chemical modification of chemical groups of the collagen, and is that reacted with a material that comprises or generates a curable group upon the reaction.

Whenever a curable collagen comprises more than one curable groups, the curable groups can be the same of different.

According to some of any of the embodiments described herein, at least a portion, or all, of the curable groups in a curable collagen of the present embodiments are photopolymerizable groups (e.g., UV-curable groups) that are capable of undergoing polymerization and/or cross-linking upon exposure to irradiation as described herein.

According to some of any of the embodiments described herein the curable group is a photocurable or photopolymerizable group (e.g., a (meth)acrylic group such as an acrylate or methacrylate).

Alternatively, or in addition, the curable group is a thiol-containing group, which provides disulfide bridge upon curing.

Alternatively, or in addition, the curable group or moiety is cured upon undergoing a chemical reaction, such as glycation or conjugation (using coupling agents such as EDC).

According to some embodiments, the curable groups comprise an amine and a carboxyl group which form peptide bonds upon curing.

According to some of any of the embodiments described herein, at least a portion, or all, of the curable groups in a curable collagen of the present embodiments are (meth)acrylic groups, as defined herein.

According to some of any of the embodiments described herein, an acrylic group such as methacrylamide can be generated by reacting an acrylate or methacrylate (e.g., acrylic acid, methacrylic acid, acrylic or methacrylic ester, acrylic or methacrylic anhydride) with an amine functional group (of, for example, lysine residues).

According to some of any of the embodiments of the present invention, the number of the curable groups in a curable collagen as described herein can determine the degree of curing (e.g., the degree of cross-linking) and can be manipulated in order to achieve a desired curing (e.g., cross-linking) degree.

According to some of any of the embodiments described herein, the curable collagen features a plurality of acrylamide or methacrylamide curable groups generated by reacting with lysine residues as described herein.

According to some of any of the embodiments described herein, the curable collagen features a plurality of acrylamide or methacrylamide curable groups substituting the amine groups of lysine residues in the collagen.

In some embodiments, at least 20%, or at least 30%, or at least 40%, or at least 50%, or at least 60%, or at least 70%, of the lysine residues in the collagen are substituted by a methacrylamide or acrylamide group. In some embodiments, the curable collagen features from 10% to 90%, or from 10% to 80%, or from 10% to 60%, or from 10 to 50%, or from 20 to 90%, or from 20 to 80%, or from 20 to 60%, or from 20 to 50%, of its lysine residues substituted by a methacrylamide or acrylamide group, including any intermediate values and subranges therebetween,

A curable collagen (e.g., rhCollagen) as described herein can be prepared by reacting a material that comprises a curable group or which generates a curable group with the collagen (e.g., rhCollagen), as described, for example, in WO 2018/225076.

The number of curable groups in the collagen (e.g., rhCollagen) can be controlled by manipulating the amount of the material reacted with the collagen (e.g., rhCollagen) for generating the curable groups.

According to some of any of the embodiments described herein, the curable collagen is a recombinant human type I collagen as described herein in any of the respective embodiments and any combination thereof.

### The Conjugate:

According to an aspect of some embodiments of the present invention there is provided a conjugate that comprises collagen and a plurality of curable elastic moieties covalently attached to the collagen,

The conjugate according to embodiments of this aspect of the present invention can be regarded as curable collagen, which, upon exposure to a curing condition, undergoes hardening, e.g., via polymerization and/or cross-linking, as described herein, by means of polymerization and/or cross-linking of at least the curable elastic moieties, and optionally also other curable groups on the collagen (in case a curable collagen is conjugated with the plurality of curable elastic moieties.

According to some of any of the embodiments described herein, the conjugate comprises collagen and a plurality of elastic moieties covalently attached to the collagen, at least a portion of the elastic moieties are elastic moieties that feature a curable group, as defined herein.

By “plurality” it is meant two or more, preferably three or more, moieties that are attached to the collagen.

The terms “elastic” and “elastomeric” as indicated herein with regard to a group (e.g., curable group) or material (e.g., curable material) are used herein interchangeably.

The plurality of elastomeric moieties can be the same or different. When different, the difference can be in the chemical composition or stereochemistry of the elastic moiety and/or in the type of the curable group and/or in the position of the curable group,

An elastic moiety that features a curable group is also referred to herein interchangeably as a “curable elastic moiety” or as a “curable elastomeric moiety” or as “an elastomeric moiety that features a curable group”, or as “an elastic moiety that features a curable group”, and all means that an elastic or elastomeric moiety features one or more curable groups.

According to some of any of the embodiments described herein, the elastomeric moiety is a moiety that confers elasticity to the hardened material formed upon polymerization and/or cross-linking of the respective curable material. Such moieties typically comprise alkyl, alkylene chains, hydrocarbon chains, alkylene glycol groups or chains (e.g., oligo or poly(alkylene glycol) as defined herein, urethane, oligourethane or polyurethane moieties, as defined herein, and the like, including any combination (e.g., co-polymers) of the foregoing.

By “elasticity” it is meant an ability of a deformed material body to return to its original shape and size when the forces causing the deformation are removed. Elasticity can be determined, for example, by the determining storage modulus, elastic modulus, and/or shear recovery rate of the hardened material, Exemplary methods of determining these parameters are described in the Examples section that follows. Other methods are well-known in the art and are also contemplated.

A curable elastomeric moiety can be a mono-functional elastomeric moiety, that comprises one curable group, or a multi-functional curable moiety, that comprises two or more curable groups.

A mono-functional curable elastomeric moiety according to some embodiments of the present invention can be derived from a vinyl-containing compound represented by Formula I:

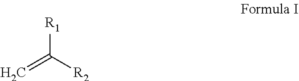

wherein at least one of R₁ and R₂ is and/or comprises an elastomeric moiety, as described herein.

The (═CH₂) group in Formula I represents a polymerizable group, and is, according to some embodiments, a UV-curable group, such that the elastomeric curable material and the moiety derived therefrom is a UV-curable material or moiety.

For example, R₁ is or comprises an elastomeric moiety as defined herein and R₂ is, for example, hydrogen, C(1-4) alkyl, C(1-4) alkoxy, or any other substituent, and is preferably hydrogen or alkyl such as methyl.

In some embodiments, R₁ is a carboxylate, and the curable elastomeric moiety is a mono-functional (meth)acrylate. In some of these embodiments, R₂ is hydrogen, and the curable elastomeric moiety is mono-functional acrylate. In some of these embodiments, R₂ is methyl, and the curable elastomeric moiety is a mono-functional methacrylate. Curable moieties in which R₁ is carboxylate and R₂ is hydrogen or methyl are collectively referred to herein as “(meth)acrylates”.

In some of any of these embodiments, the carboxylate group, —C(═O)—ORa, comprises Ra which is or comprises an elastomeric moiety as described herein, and which is linked to the collagen as described herein. In some embodiments, the Ra elastomeric moiety terminates by a reactive group that is used for conjugating a compound of Formula I to a respective group of the collagen (e.g., a carboxylate group that reacts with amine groups of lysine residues of the collagen.

In some embodiments, R₁ is amide, and the elastomeric moiety is a mono-functional acrylamide. In some of these embodiments, R₂ is hydrogen, and the curable elastomeric moiety is a mono-functional acrylamide. In some of these embodiments, R₂ is methyl, and the curable elastomeric moiety is a mono-functional methacrylamide. Curable elastomeric moieties in which R₁ is amide and R₂ is hydrogen or methyl are collectively referred to herein as “(meth)acrylamide”.

(Meth)acrylates and (meth)acrylamides are collectively referred to herein as (meth)acrylic materials.

In multi-functional elastomeric moieties, the two or more polymerizable groups are linked to one another via an elastomeric moiety, as described herein, and the elastomeric moiety is also linked to the collagen.

In some embodiments, a multi-functional elastomeric moiety can be represented by Formula I as described herein, in which R₁ comprises an elastomeric material that terminates by a polymerizable group, as described herein,

For example, a di-functional elastomeric curable moiety can be represented by Formula I*:

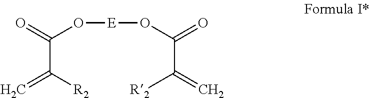

wherein E is an elastomeric linking moiety as described herein, and R′₂ is as defined herein for R₂.

In some embodiments, a multi-functional (e.g., di-functional, tri-functional or higher) elastomeric curable material can be collectively represented by Formula II:

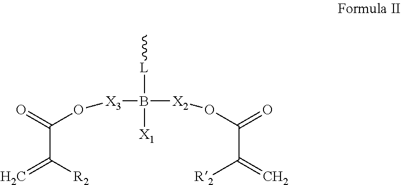

Wherein:
L represents an attachment point to the collagen, and can be a bond, or a linking moiety such as an alkylene, or a hydrocarbon chain;
R₂ and R′₂ are as defined herein;
B is a tri-functional or tetra-functional branching unit as defined herein (depending on the nature of X₁);
X₂ and X₃ are each independently absent, an elastomeric moiety as described herein, or is selected from an alkyl, a hydrocarbon, an alkylene chain, a cycloalkyl, an aryl, an alkylene glycol, a urethane moiety, and any combination thereof; and
X₁ is absent or is selected from an alkyl, a hydrocarbon, an alkylene chain, a cycloalkyl, an aryl, an alkylene glycol, a urethane moiety, and an elastomeric moiety, each being optionally being substituted (e.g., terminated) by a meth (acrylate) moiety (O—C(═O) CR″₂═CH₂), and any combination thereof, or, alternatively, X₁ is:

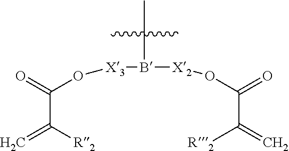

wherein:
the curved line represents the attachment point;
B′ is a branching unit, being the same as, or different from, B;
X′₂ and X′₃ are each independently as defined herein for X₂ and X₃; and
R″₂ and R′″₂ are as defined herein for R₂ and R′₂.
provided that at least one of X₁, X₂ and X₃ is or comprises an elastomeric moiety as described herein.

The term “branching unit” as used herein describes a multi-radical, preferably aliphatic or alicyclic group. By “multi-radical” it is meant that the linking moiety has two or more attachment points such that it links between two or more atoms and/or groups or moieties.

That is, the branching unit is a chemical moiety that, when attached to a single position, group or atom of a substance, creates two or more functional groups that are linked to this single position, group or atom, and thus “branches” a single functionality into two or more functionalities.

In some embodiments, the branching unit is derived from a chemical moiety that has two, three or more functional groups. In some embodiments, the branching unit is a branched alkyl or a branched linking moiety as described herein.

Multi-functional elastomeric curable materials featuring 4 or more polymerizable groups are also contemplated, and can feature structures similar to those presented in Formula II, while including, for example, a branching unit B with higher branching, or including an X₁ moiety featuring two (meth)acrylate moieties as defined herein.

In some embodiments, the elastomeric moiety, e.g., Ra in Formula I or the moiety denoted as E in Formulae I* and II, is or comprises an alkyl, which can be linear or branched, and which is preferably of 3 or more or of 4 or more carbon atoms; an alkylene chain, preferably of 3 or more or of 4 or more carbon atoms in length; an alkylene glycol as defined herein, an oligo (alkylene glycol), or a poly(alkylene glycol), as defined herein, preferably of 4 or more atoms in length, a urethane, an oligourethane, or a polyurethane, as defined herein, preferably of 4 or more carbon atoms in length, and any combination of the foregoing.

In some of any of the embodiments described herein, the elastomeric curable material is a (meth)acrylic curable material, as described herein, and in some embodiments, it is an acrylate.

In some of any of the embodiments described herein, the elastomeric curable moiety is a mono-functional elastomeric curable moiety, and is some embodiments, the mono-functional elastomeric curable material is represented by Formula I, wherein R₁ is —C(═O)—ORa or —C(═O)—NH—Ra and Ra is or comprises a poly(alkylene glycol) chain (e.g., of 4 or more, preferably 6 or more, preferably 8 or more, alkylene glycol groups), as defined herein.

In some of any of the embodiments described herein, the elastomeric curable moiety is a mono-functional elastomeric curable moiety, and is some embodiments, the mono-functional elastomeric curable material is represented by Formula I, wherein R₁ is —C(═O)—NH—Ra and Ra is or comprises a poly(alkylene glycol) chain (e.g., of 4 or more, preferably 6 or more, preferably 8 or more, alkylene glycol groups), as defined herein.

In some of any of the embodiments described herein, the elastomeric curable moiety is selected such that an elastomeric material from which it is derived provides when hardened (alone), a polymeric material that features a Tg lower than 0° C. or lower than −10° C.

According to some of any of the embodiments described herein, in each of the curable elastomeric moieties, the curable group is at a terminus of each of the elastic moiety, such that, for example, in Formula I, when Ra is or comprises an elastomeric moiety, the latter is attached at its other end to the collagen. It should be noted that curable elastomeric moieties that feature, alternatively or in addition, curable groups at a position other than the terminus are also contemplated.

According to some of any of the embodiments described herein, in at least a portion, or each, of the curable elastomeric moieties, the curable group is a photocurable or photopolymerizable group, for example, a UV-curable group.

According to some of any of the embodiments described herein, in at least a portion, or each, of the curable elastomeric moieties, the curable group is a (meth)acrylic group. In some of these embodiments, the curable group is a (meth)acrylamide, and in some embodiments, a methacrylamide.

According to some of any of the embodiments described herein, in at least a portion, or each, of the curable elastomeric moieties, the elastic moiety is or comprises a poly(alkylene glycol) moiety, as described herein, and in some of these embodiments, the poly(alkylene glycol) moiety features a (meth)acrylic group (e.g., a (meth)acrylamide) at its terminus.

In at least a portion, or each, of the elastomeric moieties, the curable group is linked to the elastomeric (e.g., poly(alkylene glycol)) moiety via a linking moiety, such that, for example, a (meth)acrylic curable elastomeric moiety is represented by Formula A:

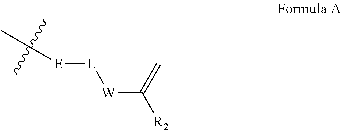

wherein:
R₂ is as defined herein in any of the respective embodiments,
W is —(C═X)—O— or —C═X—NRa;
X is O or S;
Ra is hydrogen or alkyl;
L is a linking moiety; and
E is the elastomeric moiety, for example, a poly(alkylene glycol) as defined herein in any of the respective embodiments.

The dashed line represents an attachment point to the collagen (e.g., via a covalent bond as described herein).

In some embodiments, the linking moiety is or comprises an alkylene chain, preferably a short alkylene chain of no more than 10, or no more than 8, or no more than 6, or no more than 4, carbon atoms in length, for example, of from 1 to 6, or from 1 to 4, carbon atoms in length.

In some embodiments, the linking moiety is attached to the elastomeric moiety (e.g., poly(alkylene glycol) moiety via a bond such as an amide bond, a carbamate bond, an ether bond, an ester bond, a thioester bond, a thioamide bond, a thiocarbamate bond, a sulfonamide bond, and the like. In some of these embodiments, the linking moiety is connected to the elastomeric moiety via a carbamate bond.

According to some of any of the embodiments described herein, when the elastomeric moiety is or comprises a poly(alkylene glycol) moiety, an average molecular weight of the plurality of such moieties is at least 1000 grams/mol, or at least 2000 grams/mol, or at least 3000 grams/mol or at least 4000 grams/mol, for example, in a range of from about 1000 to about 20000 grams/mol, from about 2000 to about 20000 grams/mol, from about 3000 to about 20000 grams/mol, from about 4000 to about 20000 grams/mol, from about 1000 to about 15000 grams/mol, from about 2000 to about 15000 grams/mol, from about 3000 to about 15000 grams/mol, or from about 4000 to about 15000 grams/mol, from about 1000 to about 10000 grams/mol, from about 2000 to about 10000 grams/mol, from about 3000 to about 10000 grams/mol or from about 4000 to about 10000 grams/mol, or from about 1000 to about 8000 grams/mol, from about 2000 to about 8000 grams/mol, from about 3000 to about 8000 grams/mol, from about 4000 to about 8000 grams/mol, including any intermediate values and subranges therebetween.

Each of the curable elastomeric moieties as described herein in any of the respective embodiments can be linked to the collagen by means of a covalent bond between a functional (reactive) group of the curable elastomeric moiety and a functional group of the collagen, preferably, a functional group at a collagen terminus and/or a functional group of an amino acid side chain. The curable elastomeric moieties can be linked to the collagen via the same of different bonds.

According to some of any of the embodiments described herein, at least a portion, or all, of the elastomeric moieties are covalently attached to lysine residues of the collagen.

According to some of any of the embodiments described herein, at least a portion, or all, of the elastomeric moieties are covalently attached to the collagen via a carbamate bond.

According to some of any of the embodiments described herein, at least a portion, or all, of the elastomeric moieties are covalently attached to lysine residues of the collagen via a bond such as an amide bond, a carbamate bond, a thioamide bond, a thiocarbamate bond, a sulfonamide bond, a hydrazine bond, a hydrazine bond, and the like.

According to some of any of the embodiments described herein, at least a portion, or all, of the elastomeric moieties are covalently attached to lysine residues of the collagen via a carbamate bond.

According to some of any of the embodiments described herein, at least 1%, for example, from 1 to 20, or from 1 to 10%, or at least 2%, for example, from 2 to 20%, or from 2 to 10%, of the lysine residues in the collagen have the curable elastomeric moieties covalently attached thereto (e.g., via a carbamate bond).

An exemplary conjugate according to the present embodiments is presented in FIG. 1 and is also referred to herein as “CPM”.

According to some of any of the embodiments described herein, the collagen to which the curable elastomeric moieties are attached is as described herein in any of the respective embodiments.

According to some of any of the embodiments described herein, the collagen is a human Type I collagen, as described herein.

According to some of any of the embodiments described herein, the collagen is a recombinant collagen, as described herein.

According to some of any of the embodiments described herein, the collagen is a plant-derived recombinant collagen, as described herein.

According to some of any of the embodiments described herein, the collagen is a plant-derived recombinant human Type I collagen, as described herein, for example, tobacco-derived collagen.

According to some of any of the embodiments described herein, the collagen to which the elastomeric curable groups are attached features a plurality of curable groups, e.g., photocurable groups, other than the curable elastomeric moieties, such that in some embodiments, the conjugate comprises a curable collagen as described herein in any of the respective embodiments, to which are attached curable elastomeric moieties as described herein in any of the respective embodiments and any combination thereof.

An exemplary curable collagen according to the present embodiments is also referred to herein as “CMR”.

An exemplary conjugate that comprises a curable collagen as described herein in any of the respective embodiments, to which are attached curable elastomeric moieties is also referred to herein as “CPMR”.

According to an aspect of some embodiments of the present invention, there are provided processes of preparing a conjugate as described herein.

Generally, the process is effected by coupling an elastomeric moiety that terminates at one end by a curable group as described herein and at the other end by a first reactive moiety with a collagen as described herein, whereby the contacting is effected under conditions that allows a reaction between the first reactive moiety and a chemically compatible moiety of the collagen.

According to some embodiments, the process further comprises, prior to the coupling, preparing an elastomeric moiety that terminates at one end by a curable group as described herein and at the other end by the first reactive moiety.

Exemplary synthetic pathways are described in further detail in the examples section that follows.

According to some of any of the embodiments described herein, there is provided an elastomeric curable collagen, which is a conjugate of a curable collagen as described herein in any of the respective embodiments and any combination thereof and a plurality of elastic moieties attached to the curable collagen. In some of these embodiments, the elastic moieties do not feature a curable group. The elastic moieties can be the same or different and each can independently an elastic moiety as described herein in any of the respective embodiments, In exemplary embodiments, at least a portion of the elastic moieties comprise a poly(alkylene glycol) moiety as described herein in any of the respective embodiments and any combination thereof. In some of these exemplary embodiments, at least a portion of the poly(alkylene glycol) moieties are “capped”, namely, terminate with a group other than hydroxy, for example, by an alkoxy (e.g., methoxy). In exemplary embodiments, the conjugate comprises a plurality poly(ethylene glycol) moieties, each having an average molecular weight of about 5,000 or 6,000 grams/mol, and each being terminated by a methoxy group. An exemplary conjugate according to these embodiments is described in Example 6 in the Examples section that follows, and is also referred to as “PCMR”.

### Curable Formulation:

According to an aspect of some embodiments of the present invention there is provided a formulation (or composition) that comprises a conjugate as described herein, which is also referred to herein as a curable composition or a curable formulation. According to some embodiments, the curable composition is usable in additive manufacturing of a 3D object as described herein (e.g., in bioprinting). According to some embodiments, the composition is usable, or is for use, in the preparation of, or as, one or more modeling material formulation(s) for an additive manufacturing process (e.g., bioprinting). The additive manufacturing is of a three-dimensional object that comprises, in at least a portion thereof, a collagen material as described herein.

According to some of the present embodiments, a composition that comprises a conjugate as described herein is also referred to herein as a bioink composition or a bioink formulation or simply as bioink.

According to some of any of the embodiments described herein, the curable formulation further comprises a carrier and in some embodiments, the carrier is an aqueous carrier.

The aqueous carrier can be water, a buffer featuring pH in a range of from about 2 to about 10, or from about 2 to about 9, or from about 3 to about 9, or from about 3 to about 8, a basic aqueous solution or an acidic aqueous solution.

The aqueous carrier can comprise salts and other water-soluble materials at varying concentrations. In some embodiments, a concentration of a salt in the carrier ranges from about 0.1 mM to about 0.2 M, or from about 0.1 mM to about 0.1 M, or from about 0.1 mM to about 100 mM, or from about 0.1 mM to about 50 mM, or from about 0.1 mM to about 20 mM, including an intermediate values and subranges therebetween.

In some embodiments, the aqueous carrier comprises salts at physiologically acceptable concentrations, such that the formulation features osmolarity around a physiological osmolarity. In some embodiments the aqueous carrier comprises a phosphate salt, for example, a sodium phosphate monobasic (NaH₂PO₄) and/or a sodium phosphate dibasic (sodium hydrogen phosphate; Na₂HPO₄). In some embodiments, the total concentration of the phosphate salt(s) in the formulation is about 0.1 M.

In some embodiments, the aqueous carrier comprises NaCl or any other physiologically acceptable salt.

In some embodiments, the aqueous carrier comprises a phosphate buffer and in some embodiments, the aqueous carrier comprises a phosphate buffer saline, which comprises sodium phosphate monobasic and/or sodium phosphate dibasic and NaCl.

The phosphate buffer saline (PBS) can be a commercially available PBS (e.g., DPBS) or a custom-made buffer featuring a desirable pH and/or osmolarity.

In exemplary embodiments, the aqueous carrier comprises a phosphate buffer that comprises a phosphate sodium salt as described herein at a concentration of about 0.1M and NaCl at a concentration of from about 0 mM to about 200 mM, including any intermediate value and subranges therebetween.

Any other buffers are also usable in the context of the present embodiments.

In some of any of the embodiments described herein, the aqueous carrier comprises an acid.

In some embodiments, a concentration of the acid is lower than 100 mM, and can be, for example, of from 0.1 mM to 50 mM, or from 0.1 mM to 30 mM, or from 0.1 mM to 40 mM, or from 0.1 mM to 30 mM, or from 1 to 30 mM, or from 10 to 30 Mm, including any intermediate values and subranges therebetween.

The acid can be an inorganic acid (e.g., HCl) or an organic acid, preferably which is water soluble at the above-indicated concentrations (e.g., acetic acid),

In some of any of the embodiments described herein, the aqueous carrier comprises a culturing medium. The culturing medium can be a commercially available culturing medium or a custom-made culturing medium. The culture medium can be any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, NY, USA).

The culture medium may be supplemented with various antibiotics (e.g., Penicillin and Streptomycin), growth factors or hormones, specific amino acids (e.g., L-glutamin) cytokines and the like.

According to some of any of the embodiments described herein, the curable formulation features a pH that ranges from about 2 to about 9, or from about 3 to about 9, or from about 3 to about 8.5, or from about 3 to about 8, or from about 3.5 to about 9, or from about 3.5 to about 8.5, or from about 3.5 to about 8, or from about 4 to about 8.5, or from about 4 to about 8, or from about 4.5 to about 8.5, or from about 4.5 to about 8, or from about 5 to about 8.5, or from about 5 to about 8, or from about 5.5 to about 8.5, or from about 5.5 to about 8, or from about 6 to about 8, including any intermediate values and subranges therebetween.

In some of any of the embodiments described herein, a concentration of a conjugate as described herein in any of the respective embodiments and any combination thereof in the curable formulation ranges from 0.5 mg/mL to 50 mg/mL, or from 0.5 mg/mL to 20 mg/mL, or from 1 mg/mL to 50 mg/mL, or from 1 mg/mL to 40 mg/mL, or from 1 mg/mL to 30 mg/mL, or from 1 mg/mL to 20 mg/mL, or from 0.5 mg/mL to 10 mg/mL, or from 1 mg/mL to 10 mg/mL, including any intermediate values and subranges therebetween.

A concentration of the conjugate in a curable formulation containing same can affect the rheological properties of the formulation and of the hardened material obtained upon curing (upon exposure to a curing condition such as, for example, irradiation), and can be manipulated in accordance with the AM methodology and conditions employed and desired properties of the final object or a portion thereof.

According to some of any of the embodiments described herein, the curable formulation features a shear-thinning behavior (e.g., at room temperature, for example, of from 20 to 25° C.) and is a shear-thinning composition.

The term “shear-thinning” describes a property of a fluidic material that is reflected by a decrease in its viscosity (increase in its fluidity) upon application of shear forces (under shear strain), at an indicated temperature, when determined using a rheometer as described in the Examples section that follows.

In some of the present embodiments, a shear-thinning material is such that exhibits a significant, e.g., at least 100%, reduction in its shear modulus upon increasing the shear strain from about 1% to above 50%, Shear-thinning materials therefore exhibit a shear-dependent viscosity profile.

According to some of any of the embodiments described herein, the curable formulation features a fast recovery rate upon a change in the applied shear force (a fast shear recovery).

According to some of any of the embodiments described herein, the curable formulation features a change of no more than 6%, or of no more than 10% upon shear rest (zero shear force) of 1 minute, 2 minutes, 3 minutes, 4 minutes, 5 minutes and even 10 minutes.

According to some of any of the embodiments described herein, the curable formulation features at least 80% recovery, or at least 85% recovery, or at least 90% recovery, or at least 92% recovery, of its viscosity upon increasing the shear rate from about 0 l/sec or 1 l/sec to above 50 l/sec, for a time period of at least 1 minute (e.g., from about 60 seconds to about 120 seconds, e.g., about 100 seconds).

According to some of any of the embodiments described herein, the curable formulation features a viscosity of no more than 200 centipoises, or no more than 250 centipoises, at a shear rate of 10 l/sec, at room temperature, as described herein, when determined using a rheometer as described in the Examples section that follows.

According to some of any of the embodiments described herein, the curable formulation features any of the above-indicated viscosity/rheological behavior when a concentration of the curable collagen is at least 2 mg/mL, or at least 3 mg/mL, or at least 4 mg/mL, or is in a range of from about 2 mg/mL to about 10 mg/mL, or from about 2 mg/mL to about 5 mg/mL, including any intermediate values and subranges therebetween.

According to some of any of the embodiments described herein, a concentration of the conjugate in the bioink composition can range from about 0.1 to about 20, or from 0.1 to about 10, or from about 1 to about 20, or from about 5 to about 20, or from about 8 to about 20, or from about 10 to about 20, or from about 5 to about 15, or from about 8 to about 15, or from about 8 to about 12, or from about 10 to about 15, or from about 1 to about 10, or from about 2 to about 10, or from about 2 to about 5, or from about 3 to about 10, or from about 3 to about 5, or from about 2 to about 8, or from about 2 to about 6, or from about 4 to about 10, or from about 4 to about 8, or from about 4 to about 6, mg/mL, or from about 5 to about 10, or from about 6 to about 10, or from about 8 to about 10, mg/mL, including any intermediate values and subranges therebetween.

According to some of any of the embodiments described herein, the curable formulation provides, when hardened, a hydrogel material, formed upon cross-linking of the conjugate within the aqueous carrier.

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of polymeric chains (e.g., collagen chains), within the liquid dispersing medium. The polymeric chains are inter-connected (crosslinked) by chemical bonds (covalent, hydrogen and ionic/complex/metallic bonds, typically covalent bonds),

Herein throughout, whenever polymeric chains or a polymeric material is described, it encompasses a polymeric biological materials (e.g., macromolecules) such as peptides, proteins, oligonucleotides and nucleic acids.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

According to some of any of the embodiments described herein, the bioink composition features, when hardened, storage modulus (G′) of no more than 25,000 Pa.

According to some of any of the embodiments described herein, the bioink composition features, when hardened, storage modulus (G′) that is lower by at least 5,000 Pa, or at least 6,000 Pa, or at least 8,000 Pa, or at least 10,000 Pa, than a storage modulus of a comparative bioink composition that comprises a curable collagen devoid of curable elastic moieties. According to some of these embodiments, the comparative bioink composition comprises a curable collagen in the same amount as the conjugate of the present embodiments, and the same carrier and additives.

According to some of any of the embodiments described herein, the bioink composition features, when hardened, storage modulus (G′) that ranges from about 100 Pa to about 50,000 Pa, or from about 1,000 Pa to about 50,000 Pa, or from about 100 Pa to about 40,000 Pa, or from about 1,000 Pa to about 40,000 Pa, or from about 100 Pa to about 30,000 Pa, or from about 1,000 Pa to about 30,000 Pa, or from about 100 Pa to about 25,000 Pa, or from about 1,000 Pa to about 25,000 Pa, or from about 1,000 Pa to about 20,000 Pa, or from about 100 Pa to about 20,000 Pa, or from about 1,000 Pa to about 20,000 Pa, or from about 5,000 Pa to about 30,000 Pa, or from about 5,000 Pa to about 25,000 Pa, or from about 10,000 Pa to about 30,000 Pa or from about 10,000 Pa to about 25,000 Pa, including any intermediate values and subranges therebetween.

A hydrogel, according to some embodiments of the present invention, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, cross-linked gelatin formed of, for example, gelatin methacrylate, alginate, cross-linked alginate formed of, for example, alginate methacrylate, chitosan, cross-linked chitosan formed of, for example, chitosan methacrylate, glycol chitosan, cross-linked glycol chitosan from of, for example, glycol chitosan methacrylate, hyaluronic acid (HA), cross-linked hyaluronic acid form of, for example, HA methacrylate, and other cross-linked or non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

In some embodiments, the hydrogel is porous and in some embodiments, at least a portion of the pores in the hydrogel are nanopores, having an average volume at the nanoscale range.

According to some of any of the embodiments described herein, the curable formulation further comprises one or more additional materials, including, for example, one or more additional curable materials, one or more non-curable materials and/or one or more biological components or materials.

According to some of any of the embodiments described herein, the printing media (the building material, as described herein, which may include one, two or mode modeling materials formulations, e.g., one or more bioink formulations) comprises one or more additional materials, including, for example, one or more additional curable materials, one or more non-curable materials and/or one or more biological components.

According to some of any of the embodiments described herein, the additional materials are included in the conjugate-containing curable formulation (as described herein as a bioink composition) or in one or more other modeling material formulations.

Additional curable materials that can be included in the conjugate-containing curable formulation according to the present embodiments or in one or more other modeling material formulations can be any curable material as defined herein, and is preferably a biocompatible material.

In some embodiments the additional curable material is or comprises a hydrogel, as defined herein, which can form a hardened modeling material, typically upon further cross-linking and/or co-polymerization, when exposed to a curing condition at which the cross-linking and/or co-polymerization reaction occurs. Such curable materials are also referred to herein as hydrogel curable materials or as hydrogel-forming materials,

In some of any of the embodiments described herein, a curable material is or comprises a hydrogel forming material, as defined herein, which can form a hydrogel as a hardened modeling material, typically upon cross-linking, entanglement, polymerization and/or co-polymerization, when exposed to a curing condition at which the cross-linking, polymerization and/or co-polymerization, and/or entanglement reaction occurs, Such curable materials are also referred to herein as hydrogel-forming curable materials or as gel-forming materials.

The hydrogel, according to embodiments of the present invention, can be of biological origin or synthetically prepared.

According to some embodiments of the present invention, the hydrogel is biocompatible, and is such that when a biological moiety is impregnated or accumulated therein, an activity of the biological moiety is maintained, that is, a change in an activity of the biological moiety is no more than 30%, or no more than 20%, or no more than 10%, compared to an activity of the biological moiety in a physiological medium.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly(ethylene glycol), functionalized by cross-linking (e.g., curable) groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N—(N-propyl) acrylamide), poly(methacyclic acid), poly(2-hydroxyacrylamide), poly(ethylene glycol) acrylate, poly(ethylene glycol) methacrylate, and polysaccharides such as hyaluronic acid, dextran, alginate, agarose, and the like, and any co-polymer of the foregoing,

Hydrogel precursors (hydrogel-forming materials) forming such polymeric chains are contemplated, including any combination thereof.

Hydrogels are typically formed of, or are formed in the presence of, di- or tri- or multi-functional monomers, oligomer or polymers, which are collectively referred to as hydrogel precursors or hydrogel-forming agents or hydrogen-forming materials, having two, three or more polymerizable groups. The presence of more than one polymerizable group renders such precursors cross-linkable, and allow the formation of the three-dimensional network.

Exemplary cross-linkable monomers include, without limitation, the family of di- and triacrylates monomers, which have two or three polymerizable functionalities, one of which can be regarded as a cross-linkable functional group. Exemplary diacrylates monomers include, without limitation, methylene diacrylate, and the family of poly(ethylene glycol)ₙ dimethacrylate (nEGDMA). Exemplary triacrylates monomers include, without limitation, trimethylolpropane triacrylate, pentaerythritol triacrylate, tris (2-hydroxy ethyl) isocyanurate triacrylate, isocyanuric acid tris (2-acryloyloxyethyl) ester, ethoxylated trimethylolpropane triacrylate, pentaerythrityl triacrylate and glycerol triacrylate, phosphinylidynetris (oxyethylene) triacrylate.

In some of any of the embodiments described herein, a curable material, whether monomeric or oligomeric, can be a mono-functional curable material or a multi-functional curable material.

Curable materials usable in the field of bioprinting are predominantly based on either naturally derived materials, including, for example, Matrigel, Alginate, Pectin, Xanthan gum, Gelatin, Chitosan, Fibrin, Cellulose and Hyaluronic acid, which can be isolated from animal or human tissues, or recombinantly-generated, or synthetically-prepared materials, including, for example, poly(ethyleneglycol); PEG, gelatin methacrylate; GelMA, poly(propylene oxide); PPO, poly(ethylene oxide); PEO; PEG, polyethyleneglycol-diacrylate, polyglutamic acid, PLGA/PLLA, poly(dimethyl siloxane); Nanocellulose; Pluronic F127, short di-peptides (FF), Fmoc-peptide-based hydrogels such as Fmoc-FF-OH, Fmoc-FRGD-OH, Fmoc-RGDF-OH, Fmoc-2-Nal-OH, Fmoc-FG-OH, and thermoplastic polymers such as Polycaprolactone (PCL), Polylactic acid (PLA) or Poly(D,L-lactide-co-glycolide).

Exemplary curable materials usable in the context of the present embodiments include, but are not limited to, Matrigel, Gelatin methacrylate (GelMA), Nanocellulose (nano-scaled structured materials which are UV-curable, including cellulose nanocrystals (CNC), cellulose nanofibrils (CNF), and bacterial cellulose (BC), also referred to as microbial cellulose), Pluronic® materials, including, for example, Pluronic F127 which is fluid at a low temperature forms a gel at a high temperature, above critical micellar concentration (CMC) and Pluronic F127-diacrylate (DA) which is UV-curable, Hyaluronic acid (HA), Acrylated hyaluronic acid (AHA), methacrylated hyaluronic acid (MAHA), Poly-(ethylene glycol) diacrylate (PEGDA), Alginate, Xanthan gum, Pectin, Chitosan which can be crosslinked with a chemical agent such as Glutaraldehyde, Genipin or Sodium Tripolyphosphate (TPP).

According to some of any of the embodiments described herein, the additional curable material features one or more curable groups that undergo polymerization and/or cross-linking under the same condition as the curable clastic moieties in the conjugate of the present embodiments. In some of these embodiments, the additional curable material features photocurable (e.g., UV-curable) groups, for example, acrylic groups as described herein.

According to some of any of the embodiments described herein, the additional curable material is or comprises a poly(alkylene glycol) such as a poly(ethylene glycol) that features one or more photocurable group(s), for example, one or more acrylic group(s) as described herein. In some of these embodiments, the additional curable material can be, for example, a poly(alkylene glycol) (meth)acrylate such as a poly(ethylene glycol) (meth)acrylate, and/or a poly(alkylene glycol) di (meth)acrylate such as a poly(ethylene glycol) di (meth)acrylate, and/or a copolymer that comprises the foregoing, for example, poly caprolactone (meth)acrylate and/or di (meth)acrylate/poly(ethylene glycol); poly(lactic acid) (meth)acrylate and/or di (meth)acrylate/poly(ethylele glycol); poly(actic acid co-glycolic acid) (meth)acrylate and/or di (meth)acrylate/poly(ethylene glycol), including any combination of the foregoing.

According to some of any of the embodiments described herein, the additional material is or comprises a poly(alkylene glycol) that features at least one (meth)acrylic group at its terminus, that is, it is a polymer or copolymer of poly(alkylene glycol) that features at least one (meth)acrylic group at its terminus.

According to some of any of the embodiments described herein, a poly(ethylene glycol) that features curable group(s) or a co-polymer thereof has an average molecular weight of at least 500 grams/mol or at least 700 grams/mol. In some embodiments, the average molecular weight is lower than 4,000, or lower than 3,000 or lower than 2,000, or lower than 1,000 grams/mol. In some embodiments, the average molecular weight ranges from 500 to 30,000, or from 500 to 20,000, or from 50 to 10,000, or from 500 to 5,000, or from 500 to 4,000, or from 500 to 3,000, or from 500 to 2,000, grams/mol, including any intermediate values and subranges therebetween. In some embodiments, the average molecular weight ranges from 3,000 to about 30,000, or from 3,000 to about 20,000, or from 3,000 to about 10,000, grams/mol, including any intermediate values and subranges therebetween.

According to some of any of the embodiments described herein, a concentration of the additional curable material ranges from 1 to 30, or from 1 to 20, or from 1 to 10, or from 5 to 20, or from 5 to 15, or from 10 to 20, or from 10 to 30, % by weight of the total weight of the composition, including any intermediate values and subranges therebetween.

As discussed herein, the conjugate-containing curable formulation according to any of the respective embodiments, is usable as one or more modeling material formulation(s) for an additive manufacturing process (e.g., bioprinting), as described in further detail hereinafter.

When a conjugate-containing curable formulation is described as comprising components other that a conjugate as described herein and a carrier, the additional components can be included in the same modeling material formulation or in a different modeling material formulation that is used in the additive manufacturing in combination with the conjugate-containing curable formulation.

For example, when an additional curable material other than the conjugate of the present embodiments is described, it can be included in the same modeling formulation that comprises the conjugate and/or in a different modeling material formulation.

Altogether, components that are described herein as included in a bioink composition, can be included either in the same modeling formulation or altogether within the bioprinting madia, the building material, and can be optionally divided between two or more modeling material formulations, as long as the additive manufacturing requirements are met.

According to some of any of the embodiments described herein, the printing media (building material) in general or the conjugate-containing curable formulation in particular further comprises a biological component or material other than the collagen (collectively referred to herein also as a biological material).

Biological components or materials that can be included in one or more curable (e.g., modeling material) formulations as described herein include cellular components, including, for example, culturing cells, and other cellular components such as cytokines, chemokines, growth factors; as well as other biological components such as proteins, agents that act to increase cell attachment, cell spreading, cell proliferation, cell differentiation and/or cell migration; an amino acid, peptides, polypeptides, proteins, DNA, RNA, lipids and/or proteoglycans.

Cells may comprise a heterogeneous population of cells or alternatively the cells may comprise a homogeneous population of cells. Such cells can be for example stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naïve or genetically modified.

According to one embodiment of this aspect of the present invention, the cells are mammalian in origin.

Furthermore, the cells may be of autologous origin or non-autologous origin, such as postpartum-derived cells (as described in U.S. application Ser. Nos. 10/887,012 and 10/887,446). Typically the cells are selected according to the desired application.

Suitable proteins which can be used include, but are not limited to, extracellular matrix proteins [e.g., fibrinogen, collagen, fibronectin, vimentin, microtubule-associated protein ID, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-c, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS17, tryptase-gamma, and matriptase-2] and protease substrates.

In addition, calcium phosphate materials, such as hydroxyapattite, for example, in a form of particles, can be used, including, but not limited to, nanoHA and nanoTCP. The particles size should be compatible with the dispensing heads so as to avoid clogging.

Non-curable materials, other than the biological materials as described herein, that can be included in one or more curable (e.g., modeling material) formulations as described herein can be materials that impart a certain property to the formulation or to the hardened formulation or material and to the part of the object formed thereby. Such a property can be a physical property (e.g., an optical property such as transparency or opacity, color, a spectral property, heat resistance, electrical property and the like), or a mechanical or rheological property such as viscosity, elasticity, storage modulus, loss modulus, stiffness, hardness, and the like. Alternatively, or in addition, non-curable materials can be such that provide a biological function, for example, therapeutically active agents.

Exemplary non-curable materials include thixotropic agents, reinforcing agents, toughening agents, fillers, colorants, pigments, dye substances (e.g., as described herein), etc.

An exemplary non-curable material includes titanium dioxide.

An exemplary non-curable material includes oxidized cellulose.

According to some of any of the embodiments described herein, one or more of the curable (e.g., modeling material) formulations comprises hyaluronic acid.

According to some of any of the embodiments described herein, one or more of the curable (e.g., modeling material) formulations comprises hyaluronic acid featuring a curable group as defined herein,

According to some of any of the embodiments described herein, one or more of the curable (e.g., modeling material) formulations comprises one or more biological components or materials such as, but not limited to, cells, growth factors, peptides, heparan sulfate and fibronectin.

According to some of any of the embodiments described herein, one or more of the curable (e.g., modeling material) formulations comprises one or more agents that modify a mechanical property of the formulation and/or the object, as described herein, such as, but not limited to, alginate, hyaluronic acid, fibrinogen, elastin, peptides and a thixotropic agent (e.g., Crystaline nano cellulose (CNC)), oxidized cellulose, titanium dioxide, Clay mineral and carbon nanotubes.

In some of any of the embodiments described herein the conjugate-containing curable formulation further comprises one or more additional curable materials as described herein in any of the respective embodiments.

In some of these embodiments, a weight ratio of the conjugate and the additional curable material in the formulation ranges from 20:1 to 1:2, or from 20:1 to 1:1, or from 10:1 to 1:1, or from 20:1 to 5:1, or from 15:1 to 5:1, including any intermediate value and subranges therebetween.

In some of any of the embodiments described herein the conjugate-containing curable formulation further comprises a thixotropic agent, as defined herein.

Herein throughout, the term “thixotropic” describes a property of a fluidic compound or material that is reflected by a time-dependent shear-thinning, that is its viscosity is decreased in correlation with the time at which shear forces are applied, and returns back to its original value when application of shear forces is ceased. In some of the present embodiments, a thixotropic material or agent is such that exhibits or imparts a significant, e.g., at least 100%, reduction in shear modulus under 50% strain.

In some of any of the embodiments described herein the conjugate-containing curable formulation further comprises a gel-forming agent, for example, a hydrogel-forming agent as described herein.

In some of any of the embodiments described herein the conjugate-containing curable formulation further comprises a biological component or material as described herein.

In some of any of the embodiments described herein the conjugate-containing curable formulation further comprises one or more curable or non-curable materials as described herein in any of the respective embodiments.

According to some of any of the embodiments described herein the conjugate-containing curable formulation further comprises one or more biological components such as, but not limited to, hyaluronic acid (including curable HA), cells, growth factors, peptides, heparan sulfate and/or fibronectin.

According to some of any of the embodiments described herein the conjugate-containing curable formulation further comprises one or more agents that modify a mechanical property of the formulation and/or the object, such as, but not limited to, alginate, hyaluronic acid, fibrinogen, elastin, peptides and a thixotropic agent (e.g., Crystalline nano-cellulose (CNC)).

In some of the embodiments where two or more modeling material formulations are used, two of more formulations are a conjugate-containing curable formulation as described herein, which differ from one another by the presence, type and/or concentration of an additional material that is included therein. For example, one formulation can comprise a conjugate as described herein, and another formulation can comprise a conjugate and a biological material as described herein. For example, one formulation can comprise a conjugate as described herein, and another formulation can comprise a conjugate as described herein and an additional curable material as described herein. For example, one formulation can comprise a conjugate as described herein and one additional curable material, and another formulation can comprise a conjugate as described herein and another additional curable material as described herein. For example, one formulation can comprise a conjugate as described herein and one additional curable material, and another formulation can comprise curable a conjugate as described herein and a non-curable material as described herein (e.g., a biological material or component). Any other combinations are contemplated.

In some of any of the embodiments described herein, all the curable materials in the building material are cured under the same curing condition. In some embodiments, all curable materials or curable groups are photocurable.

In some of any of the embodiments described herein, a formulation that comprises a curable material as described herein further comprises an agent that promotes curing or hardening of the curable material(s) when exposed to a curing condition.

The concentration of the agent can be determined in accordance with the concentration of the curable material(s) and the desired degree of curing (e.g., desired cross-linking degree).

### Photoinitiator:

When the curable materials are photocurable materials (e.g., UV-curable materials, for example, curable material that feature one or more (meth)acfrylic groups), the agent is a photoinitiator. The photoinitiator is selected in accordance with the curing mechanism (e.g., free-radical, cationic, etc.).

A free-radical photoinitiator may be any compound that produces a free radical on exposure to radiation such as ultraviolet or visible radiation and thereby initiates a polymerization reaction. Non-limiting examples of suitable photoinitiators include benzophenones (aromatic ketones) such as benzophenone, methyl benzophenone, Michler's ketone and xanthones; acylphosphine oxide type photo-initiators such as 2,4,6-trimethylbenzolydiphenyl phosphine oxide (TMPO) (e.g., Lithium phenyl-2,4,6-trimethylbenzoylphosphinate (LAP), 2,4,6-trimethylbenzoylethoxyphenyl phosphine oxide (TEPO), and bisacylphosphine oxides (BAPO's); benzoins and bezoin alkyl ethers such as benzoin, benzoin methyl ether and benzoin isopropyl ether and the like. Examples of photoinitiators are alpha-amino ketone, and bisacylphosphine oxide (BAPO's).

Exemplary photoinitiators include, but are not limited to, those of the Irgacure® family, riboflavin, rose Bengal, and more.

A free-radical photo-initiator may be used alone or in combination with a co-initiator. Co-initiators are used with initiators that need a second molecule to produce a radical that is active in the photocurable free-radical systems. Benzophenone is an example of a photoinitiator that requires a second molecule, such as an amine, to produce a free radical. After absorbing radiation, benzophenone reacts with a ternary amine by hydrogen abstraction, to generate an alpha-amino radical which initiates polymerization of acrylates. Non-limiting example of a class of co-initiators are alkanolamines such as triethylamine, methyldiethanolamine and triethanolamine.

Suitable cationic photoinitiators include, for example, compounds which form aprotic acids or Bronsted acids upon exposure to ultraviolet and/or visible light sufficient to initiate polymerization. The photoinitiator used may be a single compound, a mixture of two or more active compounds, or a combination of two or more different compounds, i.e. co-initiators. Non-limiting examples of suitable cationic photoinitiators include aryldiazonium salts, diaryliodonium salts, triarylsulphonium salts, triarylselenonium salts and the like. An exemplary cationic photoinitiator is a mixture of triarylsolfonium hexafluoroantimonate salts.

Non-limiting examples of suitable cationic photoinitiators include P-(octyloxyphenyl)phenyliodonium hexafluoroantimonate UVACURE 1600 from Cytec Company (USA), iodonium (4-methylphenyl) (4-(2-methylpropyl)phenyl)-hexafluorophosphate known as Irgacure 250 or Irgacure 270 available from Ciba Speciality Chemicals (Switzerland), mixed arylsulfonium hexafluoroantimonate salts known as UVI 6976 and 6992 available from Lambson Fine Chemicals (England), diaryliodonium hexafluoroantimonate known as PC 2506 available from Polyset Company (USA), (tolylcumyl) iodonium tetrakis (pentafluorophenyl) borate known as Rhodorsil® Photoinitiator 2074 available from Bluestar Silicones (USA), iodonium bis(4-dodecylphenyl)-(OC-6-11)-hexafluoro antimonate known as Tego PC 1466 from Evonik Industries AG (Germany).

According to some of any of the embodiments described herein, the photoinitiator is a free-radical photoinitiator, as described herein, for example, a photoinitiator of the acylphosphine oxide type.

According to some of any of the embodiments described herein, an amount of the photoinitiator in the formulation ranges from about 0.1 to about 10, or from about 0.1 to about 5, or from about 0.1 to about 3, or from about 0.1 to about 2, or from about 0.1 to about 1, % by weight, including any intermediate values and subranges therebetween.

The present inventors have surprisingly uncovered that there is a direct correlation between the amount of a photoinitiator and the viscosity of a formulation that contains a curable collagen as described herein (e.g., as denoted as CMR or as CPM and the photoinitiator (see, FIG. 8). More specifically, the present inventors have demonstrated that by manipulating the amount of an exemplary acylphosphine oxide type photoinitiator, the viscosity of the formulation can be controlled and thus can be suited to a desirable viscosity for a certain AM process. Without being bound by any particular theory, it is assumed that the effect on the formulation's viscosity is result of a unique interaction between the photoinitiator and the curable collagen.

According to an aspect of some embodiments of the present invention, there is provided a method of additive manufacturing a three-dimensional object that comprises in at least a portion thereof a collagen-based material, which is effected as described herein, and which further comprises, prior to dispensing a collagen-containing curable formulation, selecting an amount of a photoinitiator that provides a curable collagen-containing formulation that exhibits a viscosity that is suitable for the selected additive manufacturing, preparing the collagen-containing curable formulation with the selected amount of photoinitiator, and dispensing the formulation as described herein.

According to an aspect of some embodiments of the present invention, there is provided a method of preparing a formulation usable in additive manufacturing of a three-dimensional object that comprises in at least a portion thereof a collagen-based material, which comprises:
Selecting an additive manufacturing technique;
Determining a viscosity suitable for the additive manufacturing technique;
Determining an amount of the photoinitiator that provides the desired viscosity; and
Preparing the formulation by mixing the determined amount of the photoinitiator with a selected curable collagen (e.g., as described herein in any of the respective embodiments and any combination thereof) and optionally other components included in a curable formulation as described herein.

Herein throughout, and particularly in the context of additive manufacturing, the terms “method” and “process” are used interchangeably.

The method of the present embodiments can be performed while using a look-up table which defines the viscosity that suitable for each of the varying AM techniques, and which defines the amount of the photoinitiator that provided the desired viscosity, or alternatively, which, based on the publically available knowledge of viscosity values suitable for each AM technique, already defines the amount of the photoinitiator that is required to provide a viscosity that is suitable for each technique.

The method of preparing the curable formulation can therefore be effected by mixing an amount of the photoinitiator with a selected curable collagen (e.g., as described herein in any of the respective embodiments and any combination thereof) and optionally other components included in a curable formulation as described herein, wherein the amount is determined based on the above-described look-up table.

Preparing the formulation can be performed manually or automatically, upon calculating, manually or automatically, the amount of the photoinitiator.

According to some of any of the embodiments described herein, the photoinitiator is an acyl phosphine oxide type photoinitiator such as a 2,4,6-trimethylbenzolydiphenyl phosphine oxide (TMPO) or a salt thereof (e.g., Lithium phenyl-2,4,6-trimethylbenzoylphosphinate (LAP), 2,4,6-trimethylbenzoylethoxyphenyl phosphine oxide (TEPO), and bisacylphosphine oxides (BAPO's).

According to some of any of the embodiments of this aspect of the present invention, the collagen is a human type I collagen as described herein, preferably a recombinant human type I collagen as described herein in any of the respective embodiments.

Table 1 below is an exemplary look up table as described desired hereinabove, presenting desired viscosity values of modeling material formulations for representative AM processes, and respective amount of the photoinititator to be included in a formulation that comprises, for example, a curable collagen as described herein for CMR.

TABLE 1

| AM | viscosity | PI concentration |
|---|---|---|
| 3D inkjet printing | 40 cP or lower | 0.05%-0.1% |
| Extrusion printing | 2000 cP or above | <2% |
| SLA | 50 cP-1200 cP | 0.1%-2.0% |
| DLP | 50 cP-1200 cP | 0.1%-2.0% |

### Additive Manufacturing:

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object. According to embodiments of this aspect, formation of each layer is effected by dispensing at least one uncured building material, and exposing the dispensed building material to a curing condition to thereby form a hardened (cured) material,

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s), and optionally also uncured materials used to form a support, namely uncured support material formulations. The building material can also include non-curable materials that preferably do not undergo (or are not intended to undergo) any change during the process, for example, biological materials or components (other than a curable collagen as described herein) and/or other agents or additives as described herein.

The building material that is dispensed to sequentially form the layers as described herein is also referred to herein interchangeably as “printing medium” or “bioprinting medium”.

The building material can include one, two or more modeling material formulations, and at least one of the modeling material formulations comprises a conjugate as described herein and/or is a curable formulation as described herein in any of the respective embodiments and any combination thereof.

An uncured building material can comprise one or more modeling material formulations, and can be dispensed such that different parts of the object are made upon hardening (e.g., curing) of different modeling formulations, and hence are made of different hardened (e.g., cured) modeling materials or different mixtures of hardened (e.g., cured) modeling materials.

The method of the present embodiments manufactures three-dimensional objects in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different biological component; or each containing a different curable material; or each containing a different concentration of a curable material, and/or different support material formulations). Thus, different target locations can be occupied by different building materials (e.g., a modeling formulation and/or a support formulation, as defined herein),

The final three-dimensional object is made of the hardened modeling material or a combination of hardened modeling materials or a combination of hardened modeling material/s and support material/s or modification thereof (e.g., following curing). All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

An exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StercoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD),

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer,

In some embodiments, the receiving medium comprises a sacrificial hydrogel or other biocompatible material as a mold to embed the printed object, and is thereafter removed by chemical, mechanical or physical (e.g., heating or cooling) means. Such sacrificial hydrogels can be made of, for example, a Pluronic material or of Gelatin.

Once the uncured building material is dispensed on the receiving medium according to the 3D data, the method optionally and preferably continues by hardening the dispensed formulation(s). In some embodiments, the process continues by exposing the deposited layers to a curing condition. Preferably, the curing condition is applied to each individual layer following the deposition of the layer and prior to the deposition of the previous layer.

As used herein, the term “curing” describes a process in which a formulation is hardened. The hardening of a formulation typically involves an increase in a viscosity of the formulation and/or an increase in a storage modulus of the formulation (G′). In some embodiments, a formulation which is dispensed as a liquid becomes solid or semi-solid (e.g., gel) when hardened. A formulation which is dispensed as a semi-solid (e.g., soft gel) becomes solid or a harder or stronger semi-solid (e.g., strong gel) when hardened.

The term “curing” as used herein encompasses, for example, polymerization of monomeric and/or oligomeric materials and/or cross-linking of polymeric chains (either of a polymer present before curing or of a polymeric material formed in a polymerization of the monomers or oligomers). The product of a curing reaction is therefore typically a polymeric material and/or a cross-linked material. This term, as used herein, encompasses also partial curing, for example, curing of at least 20% or at least 30% or at least 40% or at least 50% or at least 60% or at least 70% of the formulation, as well as 100% of the formulation.

Herein, the phrase “a condition that affects curing” or “a condition for inducing curing”, which is also referred to herein interchangeably as “curing condition” or “curing inducing condition” describes a condition which, when applied to a formulation that contains a curable material, induces a curing as defined herein. Such a condition can include, for example, application of a curing energy, as described hereinafter to the curable material(s), and/or contacting the curable material(s) with chemically reactive components such as catalysts, co-catalysts, and activators.

When a condition that induces curing comprises application of a curing energy, the phrase “exposing to a curing condition” and grammatical diversions thereof means that the dispensed layers are exposed to the curing energy and the exposure is typically performed by applying a curing energy to the dispensed layers,

A “curing energy” typically includes application of radiation or application of heat.

The radiation can be electromagnetic radiation (e.g., ultraviolet or visible light), or electron beam radiation, or ultrasound radiation or microwave radiation, depending on the materials to be cured. The application of radiation (or irradiation) is effected by a suitable radiation source. For example, an ultraviolet or visible or infrared or Xenon or mercury or lamp, or LED source, can be employed, as described herein.

A curable material or system that undergoes curing upon exposure to radiation is referred to herein interchangeably as “photopolymerizable” or “photoactivatable” or “photocurable”.

When the curing energy comprises heat, the curing is also referred to herein and in the art as “thermal curing” and comprises application of thermal energy. Applying thermal energy can be effected, for example, by heating a receiving medium onto which the layers are dispensed or a chamber hosting the receiving medium, as described herein. In some embodiments, the heating is effected using a resistive heater.

In some embodiments, the heating is effected by irradiating the dispensed layers by heat-inducing radiation. Such irradiation can be effected, for example, by means of an IR lamp or Xenon lamp, operated to emit radiation onto the deposited layer.

In some embodiments, heating is effected by infrared radiation applied by a ceramic lamp, for example, a ceramic lamp that produces infrared radiation of from about 3 μm to about 4 μm, e.g., about 3.5 μm.

A curable material or system that undergoes curing upon exposure to heat is referred to herein as “thermally-curable” or “thermally-activatable” or “thermally-polymerizable”.

In some of any of the embodiments described herein, hardening the dispensed formulation(s) comprises exposing the dispensed formulation to a curing condition as described herein in any of the respective embodiments, for example, to irradiation (illumination).

In some embodiments, the exposure to a curing condition is for a short time period, for example, a time period of less than 3 minutes, less than 300 seconds, for example, of from 10 seconds to 240 seconds, or from 10 seconds to 120 seconds, to from 10 seconds to 60 seconds, including an intermediate values and subranges therebetween.

In some of any of the embodiments described herein, the method further comprises exposing the cured modeling material formulation(s) either before or after removal of a support material formulation, if such has been included in the building material, to a post-treatment condition. The post-treatment condition is typically aimed at further hardening the cured modeling material(s). In some embodiments, the post-treatment hardens a partially-cured formulation to thereby obtain a completely cured formulation.

In some embodiments, the post-treatment is effected by exposure to heat or radiation, as described in any of the respective embodiments herein.

Some embodiments contemplate the fabrication of an object by dispensing different formulations from different dispensing heads. These embodiments provide, inter alia, the ability to select formulations from a given number of formulations and define desired combinations of the selected formulations and their properties.

According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

In addition to the dispensing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for cooling the dispensed building material of for contacting it with a reagent that promotes curing.

Additionally, the AM system may include a leveling device for leveling and/or establishing the height of each layer after deposition and at least partial solidification, prior to the deposition of a subsequent layer.

According to the present embodiments, the additive manufacturing method described herein is for bioprinting a biological object.

As used herein, “bioprinting” means practicing an additive manufacturing process while utilizing one or more bio-ink formulation(s) that comprise(s) biological components, as described herein, via methodology that is compatible with an automated or semi-automated, computer-aided, additive manufacturing system as described herein (e.g., a bioprinter or a bioprinting system).

Herein throughout, the phrase “modeling material formulation”, which is also referred to herein interchangeably as “modeling formulation” or “modeling material composition” or “modeling composition”, or simply as a “formulation”, or a “composition”, describes a part or all of the uncured building material (printing medium) which is dispensed so as to form the final object, as described herein. The modeling formulation is an uncured modeling formulation, which, upon exposure to a curing condition, forms the object or a part thereof.

In the context of bioprinting, an uncured building material comprises at least one modeling formulation that comprises one or more biological components or materials (e.g., a conjugate as described herein), and is also referred to herein and in the art as “bioink” or “bioink formulation” or “bioink composition”.

In some embodiments, the bioprinting comprises sequential formation of a plurality of layers of the uncured building material in a configured pattern, preferably according to a three-dimensional printing data, as described herein. At least one, and preferably most or all, of the formed layers (before hardening or curing) comprise(s) one or more biological component(s) as described herein (e.g., a curable rhCollagen as described herein). Optionally, at least one of the formed layers (before hardening or curing) comprises one or more non-biological curable materials, and/or non-curable biological or non-biological components, preferably biocompatible materials which do not interfere (e.g., adversely affect) with the biological and/or structural features of the biological components (e.g., collagen) in the printing medium and/or bio-ink.

In some embodiments, the components in the bioink or the printing medium, e.g., non-curable and curable materials, and/or the curing condition applied to effect curing, are selected such that they do not significantly affect structural and/or functional properties of the biological components in the bio-ink or printing medium.

In some of any of the embodiments described herein, the building material (e.g., the printing medium) comprises modeling material formulation(s) (e.g., a bioink composition as described herein) and optionally support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bioink composition.

In some embodiments, a bioprinting system for effecting a bioprinting process/method as described herein is configured so as to allow formation of the layers under conditions that do not significantly affect structural and/or functional properties of the biological components in the bio-ink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

According to the present embodiments, the additive manufacturing is of a three-dimensional object featuring, in at least a portion thereof, a collagen-based material, and comprises dispensing at least one modeling material formulation to sequentially form a plurality of layers in a configured pattern corresponding to a shape of the object, wherein for at least a portion of the layers, the dispensing is of one or more modeling material formulation(s) that comprise the bioink composition as described herein in any of the respective embodiments and any combination thereof.

According to some of any of the embodiments described herein, the process further comprises exposing at least a portion of the dispensed layers to a curing condition suitable for hardening the bioink composition. In some of these embodiments, the curing condition comprises curing energy, for example, light energy (rirradiation, illumination).

According to some of any of the embodiments described herein, the for at least a portion of the layers, the dispensing is further of a modeling material formulation that comprises an agent that modifies a mechanical and/or rheological and/or physical property of the formulation and/or of a respective portion of the object,

According to some of any of the embodiments described herein, the for at least a portion of the layers, the dispensing is further of a modeling material formulation that comprises a biological material other than the collagen as described herein.

According to some of any of the embodiments described herein, the dispensing is at a temperature that ranges from −10 to 50° C., or from −4 to 50° C., or from −4 to 37° C. In some embodiments, the temperature is at least 10° C., or of at least 20° C., or of 37° C.

In some of any of the embodiments described herein, the additive manufacturing process (the bioprinting) is performed at a temperature of at least 10° C., or of at least 20° C., for example, at a temperature that ranges from about 10 to about 40° C., preferably from about 10° C. to 37° C., or from about 20° C. to 37° C., or from about 20° C. to about 30° C., or from about 20° C. to about 28° C., or from about 20° C. to about 25° C., including any intermediate values and subranges therebetween, or at room temperature, or at 37° C.

In some of any of the embodiments described herein, the above-indicated temperatures/temperature ranges are the temperatures at which the building material (e.g., at least a modeling material formulation that comprises a biological component as described herein) are dispensed, that is, a temperature of a dispensing head in the AM system and/or a temperature at which the modeling material formulation is maintained prior to passing in the dispensing head.

In some of any of the embodiments described herein, the AM process is performed without cooling the AM system (e.g., without cooling the dispensing heads and/or a modeling material formulation), to a temperature below room temperature, e.g., a temperature lower than 20° C. or lower than 10° C., or lower than 5° C. (e.g., 4° C.).

In some of any of the embodiments described herein, the AM system is devoid of means for cooling the system or a part thereof (e.g., means for cooling the dispensing heads and/or the modeling material formulation), to a temperature below room temperature, e.g., a temperature lower than 20° C. or lower than 10° C., or lower than 5° C. (e.g., 4° C.).

In some of any of the embodiments described herein, the additive manufacturing process (bioprinting) is performed while applying a shear force that does NOT adversely affect structural and/or functional properties of biological components (e.g., cells). Applying the shear force can be effected by passing the building material (e.g., at least a modeling material formulation that comprises a biological component as described herein) through the dispensing head, and is to be regarded also as subjecting the building material to shear force.

As discussed herein and demonstrated in the Examples section that follow, embodiments of the present invention allow to perform AM bioprinting processes under conditions that do not affect the functional and/or structural features of biological components included in the bio-ink (e.g., at low shear force and room temperature or a physiological temperature), while maintaining the required fluidity (a viscosity that imparts fluidity, e.g., lower than 10,000 centipoises or lower than 5,000 centipoises, or lower than 2,000 centipoises), and while further maintaining the curability of the dispensed building material. The embodiments of the present invention allow a successful operation of bioprinting using any of the known methodology, without being limited to the process parameters required for each such methodology.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and case-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

Generally, bioprinting can be effected using any of the known techniques for additive manufacturing. The following lists some exemplary additive manufacturing techniques, although any other technique is contemplated.

### 3D Inkjet Printing:

3D Inkjet printing is a commontype of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer, The most common methods for biological materials extrusion for 3D bioprinting applications are pneumatic or mechanical dispensing systems.

### Stereolythography (SLA) and Digital Light Processing (DLP):

SLA and DLP are additive manufacturing technologies in which an uncured building material in a bath is converted into hardened material(s), layer by layer, by selective curing using a light source while the uncured material is later separated/washed from the hardened material. SLA is widely used to create models, prototypes, patterns, and production parts for a range of industries including for Bioprinting. DLP differs from laser-based SLA is that DLP uses a projection of ultraviolet (UV) light (or visible light) from a digital projector to flash a single image of the layer across the entire uncured material at once. One of the key components of DLP is a digital micromirror device (DMD) chip, which is typically composed of an array of reflective aluminum micromirrors that redirect incoming light from the UV source to project an image of a designed pattern. For achieving a high-resolution structure, parameters such as the curing time of each layer, layer thickness, and intensity of the UV light should be tuned, for example, by controlling the concentration and types of the curable materials and the photoinitiator.

### Laser-Assisted Printing:

Laser-assisted printing technique, in the version adopted for 3D bioprinting, and is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate.

Laser assisted printing is compatible with a series of viscosities and can print mammalian cells without affecting cell viability or cell function. Cells can be deposited at a density of up to 10⁸ cells/ml with microscale resolution of a single cell per drop.

### Electrospinning:

Electrospinning is a fiber production technique, which uses electric force to draw charged threads of polymer solutions, or polymer melts.

According to some of any of the embodiments described herein, the additive manufacturing (bioprinting) is or comprises digital light processing (DLP), as described herein.

### The Object:

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

The term “object” as used herein throughout refers to a whole object or a part thereof.

In the context of the present embodiments, the object comprises in at least a portion thereof a collagen-based material.

By “collagen-based material” it is meant a material that comprises collagen, preferably a recombinant human collagen as described herein in any of the respective embodiments and any combination thereof.

In some of any of the embodiments described herein, the collagen-based material comprises a scaffold, for example, a hydrogel scaffold, made of a three-dimensional fibrillar network that comprises collagen (e.g., recombinant human collagen) as described herein.

In some of any of the embodiments described herein, the collagen-based material comprises polymerized and/or cross-linked (e.g., recombinant human) collagen, in which a plurality of monomeric and/or fibrillar collagen units are linked to one another to thereby form a three-dimensional network.

The three-dimension network or scaffold can be in a form of, for example, a film, a sponge, a porous structure, a hydrogel, and any other form, according to a desired need.

In some of any of the embodiments described herein, the object is in a form of a tissue or organ, which comprises, in at least a portion thereof, a collagen-based material as described herein. Such an object can be formulated in accordance with a respective 3D printing data of a desired organ or tissue, using, in addition to the curable collagen as described herein, additional curable materials and biological materials as described herein.

In some embodiments, the object is an implantable object. In some embodiments, the object is an artificial skin. In some embodiments, the object is an artificial tissue (e.g., connective tissue, or muscle tissue such as cardiac tissue and pancreatic tissue). Examples of connective tissues include, but are not limited to, cartilage (including, elastic, hyaline, and fibrocartilage), adipose tissue, reticular connective tissue, embryonic connective tissues (including mesenchymal connective tissue and mucous connective tissue), tendons, ligaments, and bone.

In some embodiments, the object is usable in, or is for use in, constructing an artificial organ or tissue.

The object can further comprise hardened materials formed of one or more of the additional curable materials as described herein in any of the respective embodiments, biological components or materials, as described herein in any of the respective embodiments, and/or non-curable materials as described herein in any of the respective embodiments.

In some embodiments, the object is in a form of a collagen scaffold or film, that can be used in research or therapeutic applications, for example, in repairing a damaged tissue, for example, upon seeding culturing cells therein, or in wound healing.

The scaffolds may be administered to subjects in need thereof for the regeneration of tissue such as connective tissue, muscle tissue such as cardiac tissue and pancreatic tissue.

The films can be used to construct biomedical devices such as, for example, collagen membranes for hemodialysis.

According to some embodiments, films or scaffolds can be used in cell cultures.

The phrase “cell culture” or “culture” as used herein refers to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms.

In some embodiments, the films or scaffolds can be used in a wound healing process.

In some embodiments, collagen films provided herein are used to prevent adhesions following tendon injuries, to lengthen levator palpebrae muscles ophthalmic surgery, and to repair transected nerves. Collagen films provided herein may further be used for burn dressings and in healing of bone defects.

The object of the present embodiments comprises a myriad of other uses including, but not limited to, in the treatment of diseases such as interstitial cystitis, scleroderma, and rheumatoid arthritis cosmetic surgery, as a healing aid for burn patients, as a wound-healing agent, as a dermal filler, for spinal fusion procedures, for urethral bulking, in duraplasty procedures, for reconstruction of bone and a wide variety of dental, orthopedic and surgical purposes.

### Kits:

According to an aspect of some embodiments of the present invention, there is provided a kit that comprises a conjugate as described herein in any of the respective embodiments.

According to some embodiments, the kit comprises a curable formulation (e.g., a bioink composition) that comprises the conjugate as described herein in any of the respective embodiments.

According to some embodiments, the curable formulation comprises the conjugate in a lyophilized form.

According to some embodiments, the curable formulation comprises the conjugate and an aqueous solution or carrier, as described herein.

According to some of any of the embodiments described herein, the kit is identified for use, or is usable, as a modeling material formulation for additive manufacturing (e.g., bioprinting) of an object as described herein in any of the respective embodiments.

According to some of any of the embodiments described herein, the kit further comprises an aqueous carrier, as described herein in any of the respective embodiments. In some embodiments, the conjugate or the composition and the aqueous carrier are packaged individually within the kit.

Alternatively, the kit includes instructions to prepare a modeling material formulation as described herein, by mixing the conjugate or the composition with the aqueous carrier.

The kit may further comprise other components that can be included in a bioink composition or a modeling material formulation(s) as described herein in any of the respective embodiments and any combination thereof.

The kit may further comprise instructions how to use the conjugate or the bioink composition or formulation in an additive manufacturing process as described herein.

### Dye Substance:

According to some of any of the embodiments described herein, a bioink composition or a curable (e.g., modeling material) formulation as described herein can comprise a dye substance that is capable of absorbing light at a desired wavelength range. The addition of absorbing dye substances is typically required for digital light processing bioprinting, in order to improve the resolution and achieve defined porosity and channels of scaffolds.

According to some of any of the embodiments described herein, the dye substance is such that is capable of absorbing light at a wavelength of from 300 nm to 800 nm, or from 300 nm to 600 nm, or from 300 nm to 500 nm, or from 300 nm to 450 nm, or from 350 nm to 450 nm, or preferably from 365 nm to 405 nm.

A dyes substance as described herein is also referred to herein throughout as a photoabsorber or as a photoblocker.

Exemplary dye substances that suitable for use in the context of these embodiments include, but are not limited to, food dyes, tartrazine, Sunset Yellow FCF (Yellow No. 6), Brilliant Blue FCF (FD&C Blue No. 1), indigo carmine (FD&C Blue No. 2), Fast Green FCF (FD&C Green No. 3) anthocyanins, anthocyanidin, erythrosine (FD&C Red No. 3), Allura Red AC (FD&C Red No. 40), riboflavin (Vitamin B2, E101, E101a, E106), ascorbic acid (vitamin C), Quinoline Yellow WS, carmoisine (azorubine), Ponceau 4R (E124), Patent Blue V (E131), Green S (E142), Yellow 2G (E107), Orange GGN (E111), Red 2G (E128), caramel color, phenol red, methyl orange, 4-nitrophenol, and NADH disodium salt, curcumin (E100), turmeric, alpha carotene, beta carotene, canthaxanthin (keto-carotenoid), cochineal extract, paprika, saffron, ergocalciferol (vitamin D2), cholecalciferol (vitamin D3), Citrus Red 2, annatto extract, avobenzone, 2,5-bis(5-tert-butyl-benzoxazol-2-yl)thiophene (Benetex OB+), disodium 4,4′-bis(2-sulfonatostyryl) biphenyl (Benetex OB-M1), benzenepropanoic acid (BLS 99-2), 2,3,6,7-tetrahydro-9-methyl-1H,5H-quinolizino (9,1-gh) coumarin (Coumarin 102), Martius Yellow, morin hydrate, nitrofurazone, 2-nitrophenyl phenyl sulfide (NPS), 5,12-naphthacenequinone (NTAQ), octocrylene, phenazine, 1,4-bis-(2-(5-phenyloxazolyl))-benzene (POPOP), Quinoline Yellow, 3,3′,4′,5,6-pentahydroxyflavone (Quercetin), salicylaldehyde, Sudan I, triamterene, UV386A, 1-phenylazo-2-naphthol (sudan I), 1-(2,4-dimethylphenylazo)-2-naphthol (sudan II), 1-(4-(phenyldiazenyl)phenyl) azonaphthalen-2-ol (sudan III), 1-[{2-methyl-4-[(2-methylphenyl)diazenyl}phenyl]diazenyl]naphthalen-2-ol (sudan IV), 2,5-bis(5-tert-butyl-benzoxazol-2-yl)thiophene, fluorescein, poly(3-hexylthiophene-2,5-diyl), oligothiophenes, tri-phenylamines, diketopyrrolopyrroles derivatives, 2,5-dihydro-3,6-di-2-thienyl-pyrrolo[3,4-c]pyrrole-1,4-dione, borondipyrromethenes derivatives, 1,3,5,7-tetramethyl-8-phenyl-4,4-difluoroboradiazaindacene, 2,2′-(2,5-thiophenediyl) bis(5-tert-butylbenzoxazole), (±)-a-tocopherol, 2-phenyl-2H-benzotriazole derivatives, 2,2-dimethyl-1,3-dihydroperimidin-6-yl)-(4-phenylazo-1-naphthyl) diazene (sudan black B), 1-(2-methoxyphenylazo)-2-naphthol (sudan red G), 2,5-bis(5-tert-butyl-benzoxazol-2-yl)thiophene, 4-methoxyphenol, butylated hydroxytoluene, 2-hydroxyphenyl-s-triazine, 2-(2H-benzotriazol-2-yl) phenol, and any combination thereof.

As explained in the Examples section that follows, the use of a conjugate as described herein allow using such dye substances without the risk of precipitation of the collagen.

According to some embodiments of the present invention, the dye substance has a plurality of negatively-charged groups (functional groups that are ionizable at physiological pH or at the pH of the bioink composition comprising same). Exemplary negatively charged groups include, but are not limited to, hydroxy, sulfate, sulfonate, thiol, phosphate, phosphonate, and the like.

Exemplary dye substances that feature a plurality of negatively charged groups are polysulfate dyes.

As further demonstrated in the Examples section that follows, the present inventors have uncovered that vitamin B12 can be successfully utilized as a dye substance in a bioink composition as described herein.

According to some of any of the embodiments described herein, the dye substance is Vitamin B12.

According to some of any of the embodiments described herein, the dye substance is a quinoline.

According to some of any of the embodiments described herein, the dye substance is minocycline.

According to some of any of the embodiments described herein, an amount of the dye substance ranges from 0.01 to 5%, or from 0.01 to 2%, or from 0.01 to 1%, or from 0.01 to 1%, or from 1 to 5%, or from 0.1 to 5%, or from 0.1 to 2%, or from 0.01 to 2%, or from 0.1 to 1%, or from 1 to 3%, by weight of the total weight of the formulation or composition comprising same, including any intermediate values and subranges therebetween.

According to some of any of the embodiments described herein, the substance is minocycline and its amount ranges from 0.01 to 5%, or from 0.01 to 2%, or from 0.01 to 1%, or from 0.05 to 1.5%, by weight of the total weight of the formulation or composition comprising same, including any intermediate values and subranges therebetween.

According to an aspect of some embodiments of the present invention, there is provided a curable formulation for use in additive manufacturing of a three-dimensional object, which comprises a photocurable biological material and a dye substance capable of absorbing light at a wavelength of from 300 nm to 800 nm. According to embodiments of this aspect of the present invention, the dye substance is or comprises Vitamin B12.

According to some of any of the embodiments described herein for this aspect of the present invention, an amount of the vitamin B12 ranges from 0.01 to 5%, or from 1 to 5%, or from 0.1 to 5%, or from 0.1 to 2%, or from 0.01 to 2%, or from 0.1 to 1%, or from 1 to 3%, by weight of the total weight of the formulation or composition comprising same, including any intermediate values and subranges therebetween.

The photocurable biological material can be any biological material as described herein, which features a plurality of photocurable groups as described herein, for example, (meth)acrylic groups. Examples include, but are not limited to, (meth)acrylated gelatin, (meth)acrylated hyaluronic acid, (meth)acrylated hyaluronic acid, and (meth)acrylated collagen, such as a curable collagen as described herein in any of the respective embodiments, and/or a conjugate as described herein in any of the respective embodiments.

According to an aspect of some embodiments of the present invention, there is provided a process of additive manufacturing a three-dimensional object featuring, in at least a portion thereof, a biological material, as described herein in any of the respective embodiments. The additive manufacturing is a bioprinting process as described herein in the context of a bioink composition, and comprises dispensing at least one modeling material formulation to sequentially form a plurality of layers in a configured pattern corresponding to a shape of the object.

According to embodiments of this aspect of the present invention, for at least a portion of the layers, the dispensing is of a modeling material formulation that comprises a bioink composition that comprises vitamin B12 as a sye substance, as described herein in any of the respective embodiments.

According to some embodiments, the additive manufacturing is DLP.

According to some embodiments, the process further comprises exposing the portion of the layers to irradiation suitable for hardening the bioink composition. According to exemplary embodiments, the irradiation is at a wavelength as described herein.

It is expected that during the life of a patent maturing from this application many relevant curable biocompatible materials, curable biological materials, bioprinting media, and/or bioprinting technologies will be developed and the scope of embodiments related to urable biocompatible materials, bioprinting media, and/or bioprinting technologies is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Herein throughout, whenever “centipoise” or “Cp” is indicated, the corresponding Pa· second value (1 Pa·second=1,000 centipoise) is encompassed.

Herein throughout, whenever the phrase “weight percent”, or “% by weight” or “% wt.”, is indicated in the context of embodiments of a formulation (e.g., a modeling formulation, a curable formulation, a bioink composition), it is meant weight percent of the total weight of the respective uncured formulation.

Herein throughout, an acrylic material is used to collectively describe material featuring one or more acrylate, methacrylate, acrylamide and/or methacrylamide group(s).

Similarly, an acrylic group is used to collectively describe curable groups which are acrylate, methacrylate, acrylamide and/or methacrylamide group(s), preferably acrylate or methacrylate groups (referred to herein also as (meth)acrylate groups).

Herein throughout, the term “(meth)acrylic” encompasses acrylic and methacrylic materials,

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 30, and is preferably lower, e.g., from 1 to 10, or from 1 to 6, or from 1 to 4. A hydrocarbon can be a linking group or an end group.

As used herein, the term “amine” describes both a-NR′R″ group and a-NR′-group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group or is or part of a linking moiety.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system, Representative examples are piperidine, piperazine, tetrahydrofurane, tetrahydropyrane, morpholino, oxalidine, and the like.

The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “halide” and “halo” describes fluorine, chlorine, bromine or iodine.

The term “haloalkyl” describes an alkyl group as defined above, further substituted by one or more halide.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein. The term alkoxide describes —R′O⁻ group, with R′ as defined herein.

The term “aryloxy” describes both an —O-aryl and an —O-heteroaryl group, as defined herein.

The term “thiohydroxy” or “thiol” describes a —SH group. The term “thiolate” describes a —S⁻ group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “acyl halide” describes a —(C═O)R″″ group wherein R″″ is halide, as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein,

The term “O-thiocarboxylate” describes a —OC(═S)R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′— end group or a —OC(═O)—NR′— linking group, as these phrases are defined hereinabove, with R′ and R″ as defined herein.

The term “O-carbamate” describes an —OC(═O)—NR′R″ end group or an —OC(═O)—NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “carbamate” as used herein encompasses N-carbamate and O-carbamate.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″—linking group, as these phrases are defined hereinabove, where R′ and R″ are as defined herein and R″ is as defined herein for R′ and R″.

The term “thiourea”, which is also referred to herein as “thioureido”, describes a —NR′—C(═S)—NR″R′″ end group or a —NR′—C(═S)—NR″—linking group, with R′, R″ and R′″ as defined herein.

The term “amide” as used herein encompasses C-amide and N-amide.

The term “C-amide” describes a —C(═O)—NR′R″ end group or a —C(═O)—NR′—linking group, as these phrases are defined hereinabove, where R′ and R″ are as defined herein.

The term “N-amide” describes a R′C(═O)—NR″—end group or a R′C(═O)—N—linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo (alkylene glycol). When y is higher than 4, it is a poly(alkylene glycol). Capped poly(alkylene glycol) has R′″ which is other than hydrogen and which can be, for example, an alkyl (e.g., lower alkyl), a carbonyl, and like moieties.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

Syntheses of PEG featuring a reactive group and a curable group

### Synthetic Procedure I

### Preparation of Succinimide-Capped Poly(Ethylene Glycol) (PEG) (Compound 1)

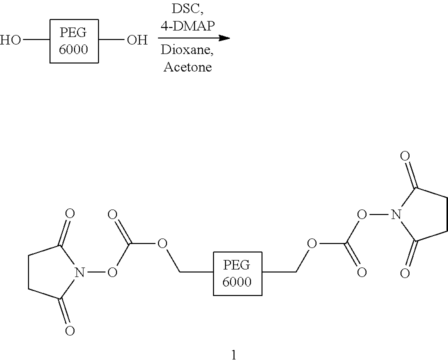

PEG 6000 (TCI America) (10 grams; 1.66 mmol) was suspended in 40 mL dry 1,4-Dioxane and the suspension was heated to 50° C. and stirred until complete dissolution. The obtained solution was allowed to cool to room temperature and N,N′-disuccunimidyl carbonate (DSC) powder (2.56 grams; 10 mmol) was then added under nitrogen stream.

4-(Dimethylamino)pyridine (4-DMAP; 1.23 gram; 10 mmol) was dissolved in acetone (30 mL) and the solution was added in one portion to the suspension. The suspension was stirred under nitrogen at room temperature overnight.

The obtained clear solution was evaporated to approximately 30 mL and was poured into 150 mL of cold diethyl ether. The precipitated solid was filtered via Büchner and was suspended in ethyl acetate for 10 minutes. The obtained white solid was then filtered via Büchner, using Whatmann filter paper No. 4 to afford Compound 1 as a white solid (9.34 gram). The product was dried under high vacuum and stored at −20° C. under argon.

¹H NMR (CDCl₃, 500 mHz, δ ppm): 4.41 (dd, 4H), 3.58 (s), 2.81 (s, 8H).

### Preparation of Methacrylated PEG (Compound 2)

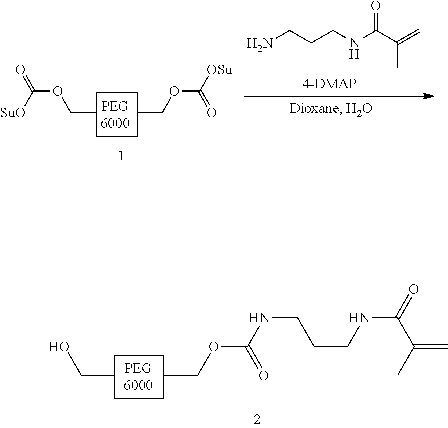

Compound 1 (9.34 grams; 1.48 mmol) was suspended in 75 mL 1,4-Dioxane. The suspension was heated to 50° C. and stirred until complete dissolution. The solution was allowed to cool to room temperature. 3-Amionopropyl methacrylamide hydrochloride (265.3 mg; 1.48 mmol) was dissolved in 10 mL of mQ water, 4-DMAP (366 mg; 2.97 mmol) was added thereto and the obtained aqueous solution was stirred until complete dissolution. The pH of the solution was 10.15. The aqueous solution was added dropwise into the Compound 1 solution. After the addition of 4 mL the solution became hazy. 3 mL of mQ water were added to facilitate dissolution. Dripping was continued over 30 minutes, and the solution was further stirred at room temperature overnight. TLC (using ninhydrin reagent as a developer) indicated no presence of a free amine. Toluene (200 mL) was added and the flask was fitted with a dean-Stark apparatus. The reaction was refluxed until no more water distilled out.

The Dean stark apparatus was removed and the volume of toluene was reduced by a rotavaporizer to about 30 mL The solution was then poured into 180 mL of cold diethyl ether, the resulting precipitate was filtered via Büchner and the white solid was re-dissolved in 30 mL dichloromethane (DCM) and was precipitated by the addition of 180 cold diethyl ether. The resulting white solid was filtered via Büchner, using Whatmann filter paper No. 4 to afford Compound 2 as a white solid (7.32 grams). The product was dried under high vacuum and stored at −20° C. under argon.

¹H NMR (CDCl₃, 500 mHz, δ ppm): 6.62 (m, 1H) 5.74 (s, 1H) 5.33 (s+m, 2H) 4.21 (m, 2H) 3.37 (dd, 2H) 3.24 (dd, 2H) 1.98 (S, 3H) 1.69 (q, 2H).

### Preparation of Nitrobenzoyl-Capped Methacrylated PEG (Compound 3)

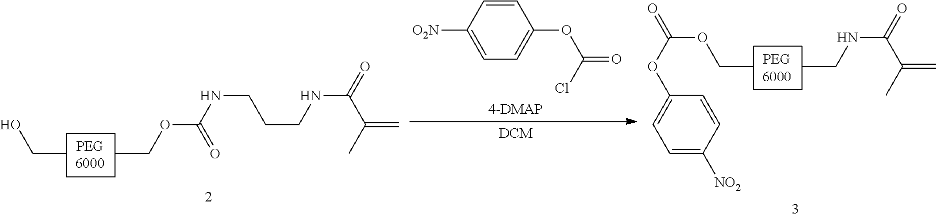

Compound 2 (7.29 grams; 1.17 mmol) was dissolved in 25 mL dry DCM.

4-Nitrobenzoyl chloride (237 mg; 1.17 mmol) was dissolved in 2 mL dry dichloromethane (DCM) and the solution was added to the Compound 2-containing solution in one portion.

4-DMAP (145 mg; 1.17 mmol) was dissolved in 2 mL dry DCM and the solution was added to the reaction mixture. The resulting solution was left to stir at room temperature overnight under nitrogen atmosphere, and was thereafter poured into 180 mL diethyl ether. The resulting white precipitate was filtered via Büchner, using Whatmann filter paper No. 1 and was then recrystallized in 150 mL ethyl acetate, filtered via Büchner and suspended in ethyl acetate for 10 minutes. The white solid was filtered via Büchner, using Whatmann filter paper No. 4 to afford Compound 3 as a white solid (6.63 grams). The product was dried under vacuum and stored at-20° C. under argon.

¹H NMR (CDCL, 500 mHz, δ ppm): 8.29 (d, 2H) 7.38 (d, 2H) 6.62 (m, 1H) 5.74 (s, 1H) 5.33 (s+m, 2H) 4.45 (m, 4H) 4.21 (m, 4H) 3.36 (dd, 2H) 3.24 (dd, 2H) 1.98 (s, 3H) 1.66 (q, 2H). * A singlet at 2.89 represent residual unreacted NHS carbonate.

### Synthetic Procedure II

### Preparation of Nitrobenzoyl-Protected PEG (Compound 11)

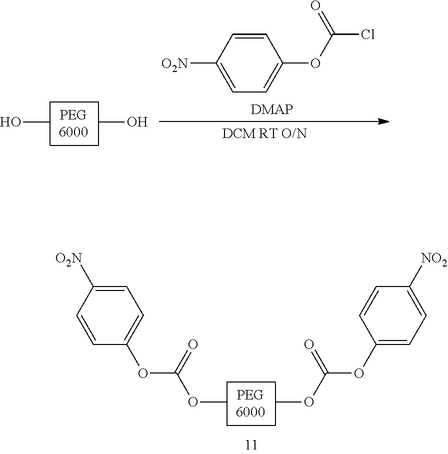

PEG 6000 (20 grams; 3.33 mmol) was dried in a vacuum oven at 120° C. at 0.1 mbar overnight, was thereafter allowed to cool to room temperature and dissolved in 50 mL dry DCM.

4-Nitrophenyl chloroformate (1.34 gram; 6.66 mmol) was dissolved in 5 mL dry DCM and the solution was added in one portion to the PEG solution, and the obtained reaction mixture was stirred for 5 minutes.

4-DMAP (821 mg; 6.66 mmol) was dissolved in 5 mL DCM, the solution was added dropwise to the reaction mixture, which was thereafter left to stir at room temperature overnight.

The solvent volume was reduced to about 30 mL by evaporation and the remaining solution was poured into 200 mL dietheyl ether. The precipitated solid was collected via filtration on a Buchner funnel and was re-dissolved in 40 mL DCM and poured into 200 mL diethyl ether. The obtained solid was collected via Buchner filtration and dried under vacuum to afford Compound 1 (19.35 grams) as a white solid.

¹H NMR (CDCl₃, 500 mHz, δ ppm): 8.20 (d, 2H), 7.32 (d, 2H), 4.37 (m, 2H), 3.57 (s, 545H).

### Preparation of Methacrylated PEG (Compound 12)

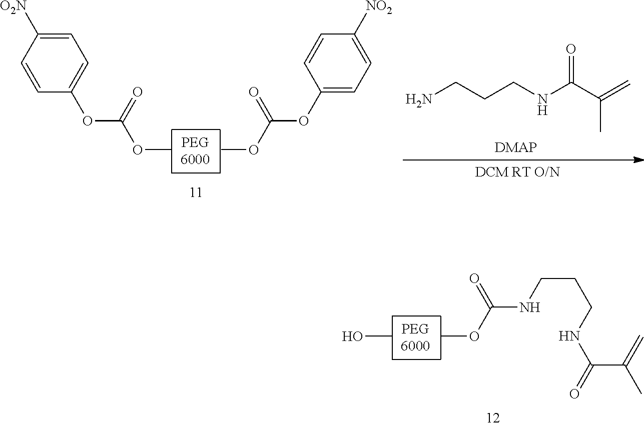

Compound 11 (19.35 grams; 3.03 mmol) was dissolved in 180 mL acetonitrile. 3-Aminopropyl methacrylate HCl (544 mg; 3.05 mmol) was dissolved in 25 mL mQ water, 4-DMAP (752 mg; 6.10 mmol) was then added and the solution was stirred until complete dissolution. The resulting solution was then added dropwise, over 1 hour, to the Compound 11 solution and the resulting reaction mixture was left to stir at room temperature. The solvent volume was thereafter reduced by evaporation to about 80 mL and 150 mL of DCM were added, The solution was dried by addition of MgSO₄, filtered and the solvent volume was reduced to about 30 mL. The solution was poured into 180 mL diethyl ether and the precipitate was collected via Buchner filtration. The white solid was re-dissolved in 50 mL DCM, the solution was poured into 200 mL diethyl ether and the resulting white solid was collected via filtration to yield Compound 12 as a white solid (16.766 grams).

¹H NMR (CDCl₃, 500 mHz, 8 ppm): 5.70 (s, 1H), 5.27 (s, 1H), 4.15 (m, 2H), 3.57 (s, 545H), 3.30 (dd, 2H), 3.17 (dd, 2H), 1.91 (s, 3H), 1.62 (q, 2H).

### Preparation of Nitro-Benzoyl-Capped Methacrylated PEG (Compound 3)

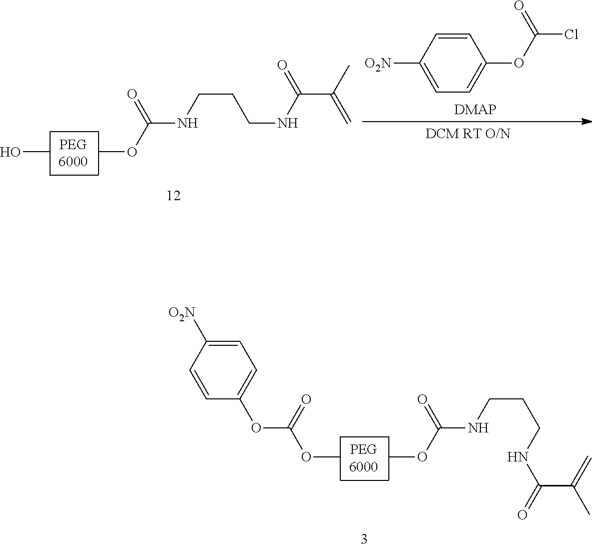

Compound 12 (16.76 grams; 2.71 mmol) was dissolved in 50 mL dry DCM. 4-Nitrophenyl chloroformate (655 mg; 3.25 mmol) was dissolved in 5 mL dry DCM and the solution was added in one portion to the reaction mixture, which was subsequently stirred for 5 minutes.

4-DMAP (334 mg; 2.71 mmol) was dissolved in 5 mL DCM and the solution was added dropwise to the reaction mixture, which was thereafter left to stir at room temperature overnight.

The solvent volume was reduced to about 30 mL by evaporation and the solution was poured into 200 mL dietheyl ether. The precipitated solid was collected via filtration on a Buchner funnel, was re-dissolved in 40 mL DCM and poured into 200 mL diethyl ether. The obtained solid was collected via Buchner filtration and dried under vacuum to afford Compound 3 as a white solid (about 14 grams).

¹H NMR (CDC), 500 mHz, δ ppm): 8.21 (d, 1H), 7.33 (d, 1H), 5.70 (s, 1H), 5.27 (s, 1H), 4.38 (dd, 1H) 4.15 (m, 2H), 3.57 (s, 545H), 3.30 (dd, 2H), 3.17 (dd, 2H), 1.91 (s, 3H), 1.62 (q, 2H).

### Synthetic Procedure III

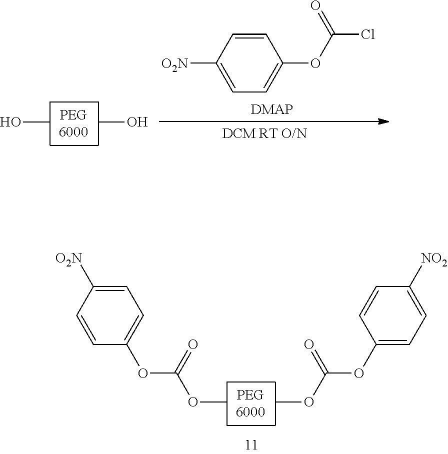

PEG 6000 (20 grams; 3.33 mmol) was placed in a 500 mL amber round bottomed flask and 240 mL of toluene were added. The flask was fitted with a Dean-Stark apparatus and residual water were distilled out for 4 hours.

The solution was allowed to cool to room temperature. 4-Nitrophenyl chloroformate (2.68 grams; 13.32 mmol) was added directly, along with triethylamine (1.85 mL; 13.32 mmol), and the reaction mixture was heated to 60° C. overnight. The solvent volume was reduced to about 30 mL by evaporation and the remaining solution was poured into 200 mL dietheylether. The precipitated solid was collected via filtration on a Buchner funnel and was re-dissolved in 40 mL DCM and poured into 200 mL diethyl ether. The obtained solid was collected via Buchner filtration and dried under vacuum to afford Compound 11 (19.35 grams) as a white solid.

¹H NMR (CDCl₃, 500 mHz, δ ppm): 8.20 (d, 4H), 7.32 (d, 4H), 4.37 (m, 4H), 3.57 (s, 545H).

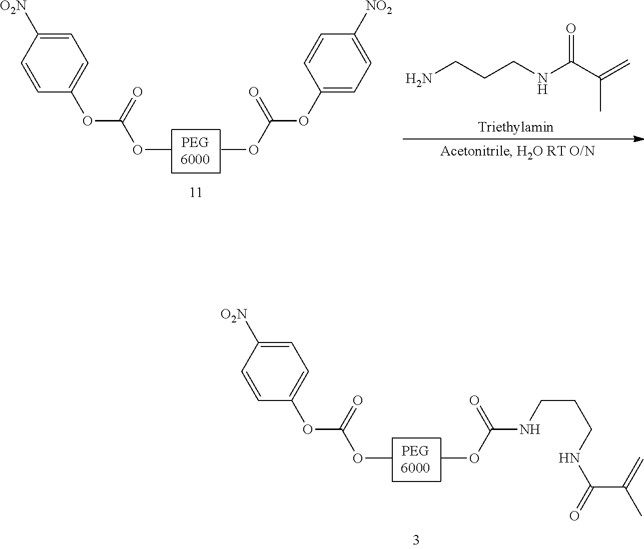

Compound 11 (19.35 grams; 3.03 mmol) was dissolved in 160 mL acetonitrile in a 250 mL round bottom flask with amber glass. 3-Aminopropyl methacrylate HCl (544 mg; 3.05 mmol) was dissolved in 5 mL mQ water, Triethylamine (850 μL; 6.10 mmol) was then added and the solution was stirred until complete dissolution. The resulting aqueous solution was then added dropwise, over 2 hours, to the Compound 11 solution and the resulting reaction mixture was left to stir at room temperature overnight. The solvent volume was thereafter reduced by evaporation to about 30 mL and 40 mL of toluene were added. The flask was fitted with a dean stark apparatus and 5 mL water were distilled out. The solution was then evaporated to reduce solvent volume to about 30 mL and was poured into 200 mL of cold diethylether. The precipitate was collected via Büchner filtration. It was then re-dissolved in 40 mL toluene and poured into 300 mL cold diethylether. Compound 3 was collected via Büchner filtration and dried under vacuum overnight to acquire 15.75 grams as a white solid.

¹H NMR (CDCl₃, 500 mHz, δ ppm): 8.24 (d, 2H), 7.36 (d, 2H), 5.70 (s, 1H), 5.31 (s, 1H), 4.38 (dd, 2H) 4.15 (m, 2H), 3.57 (s, 545H), 3.30 (dd, 2H), 3.17 (dd, 2H), 1.95 (s, 3H), 1.65 (q, 2H).

### Example 2

### Collagen-Methacrylated PEG Conjugate

### Synthesis:

727 mL of recombinant human Type I Collagen (Collplant Ltd.) (2.9 mg/mL) were placed in a 1 Liter singe jacketed reactor which was pre-cooled to 4° C., and 80 mL of MOPS 2M buffer (pH 8.0) were added thereto along with 34 mL 4M NaCl. The solution was titrated to pH 8.0 using 10N NaOH solution and was thereafter cooled to 6° C. 9.3 grams of Compound 3 (1:2 mol ratio to lysine residues of the Collagen) were dissolved in 10 mL mQ water. The resulting solution was added in one portion to the cooled (6° C.) collagen solution while stirring and the reaction mixture was left to stir at 6° C. for 18 hours. The reaction was then quenched by adding 150 mL HCl IM to obtain a solution with a pH of 3. The product was purified via dialysis which was carried out with eleven 800 mL portions of 10 mM HCl, by adding 800 mL of 10 mM HCl and reducing the volume in the vessel to 800 mL, repeatedly. The solution was thereafter concentrated to the void volume of 65 mL.

FIG. 1 presents an illustration of the reaction product of Compound 3 (PEG-MA) with the collagen. The PEG-methacrylate moieties are attached via a carbamate bond to lysine residues of the collagen).

FIG. 2 presents SDS-PAGE comparing collagen and CPM.

The SDS-PAGE analysis suggests that at least 2% of the lysine residues, e.g., 2-10% of the lysine residues, have a PEG-MA moiety attached thereto.

### Characterization:

Three formulations, each containing 7% PEG-DA 3400 and Lithium phenyl-2,4,6-trimethylbenzoylphosphinate (LAP) as a photoinitiator were prepared. One of the formulations further contained methacrylated recombinant hymen type I collagen (CMR) at a 4 mg/mL concentration; one of the formulations further contained the reaction product of Compound 3 (PEG-MA) with the collagen such as shown in FIG. 1, also referred to as “CPM”, at a 4 mg/mL concentration; and one served as control, with no protein.

Viscosity measurements were performed using Cone-Plate (40 mm 1°) UHP Steel geometry, with sample conditioning at 22° C., 10 seconds soak time, pre-shear in 2.0 rad/sec for 20 seconds. Following that, Flow Sweep (logarithmic) test was applied with 0.01 to 1000 l/sec shear rate, 3 points per decade. For steady-state sensing max equilibrium time 60 seconds was used, sample period 5 seconds with 5% tolerance. The applied forces were removed for 1, 3 and 5 minutes after initial measurement, then viscosity was re-measured. Sample volume: 300 microliters, containing CPM or CMR at 9.3 mg/mL in a 10 mM HCl buffer solution pH 2.5

Then obtained data is presented in FIGS. 3A (for a CPM formulation) and 3B (for a CMR formulation).

As can be seen, the CPM samples retain their viscosity even after 1 minute of relaxation where the viscosity of CMR drops significantly and does not recover even after 5 minutes relaxation.

Recovery under shear (Shear Recovery) was tested as follows: Flow Peak measurements were performed using Discovery HR 2 [TA Instruments] with Cone-Plate (40 mm 1°) UHP Steel geometry, with sample conditioning at 22° C., 10 seconds soak time, pre-shear in 2.0 rad/sec for 20 seconds, Then, Flow Peak Hold (logarithmic) test was performed at shear rate 1.0 l/sec for 200 seconds, followed by shear rate 100 l/sec for 100 seconds, followed by shear rate 1.0 l/sec for 100 seconds. Sample volume: 300 microlites.

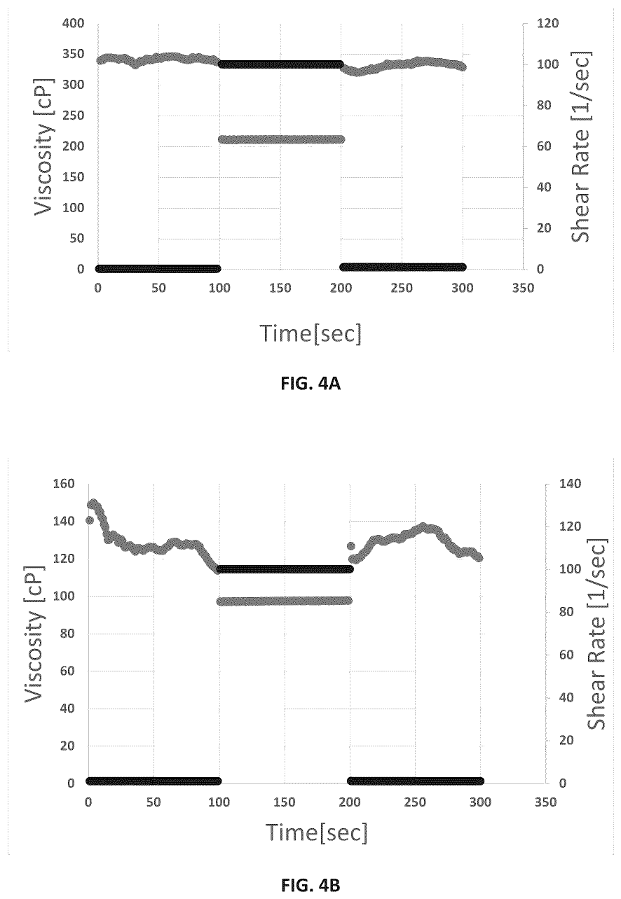

FIGS. 4A-B present the data showing the change in viscosity upon manipulating shear force for a formulation comprising CPM (FIG. 4A) and for a formulation comprising CMR (FIG. 4B), as described hereinabove and show that while CPM features a viscosity higher than that of CMR, its recovery under shear is higher (97% for CPM versus 87% for CMR), indicating that the PEGylated collagen withstands shear forces significantly better than CMR.

The photo-rheological characterization of all the scaffolds which were obtained upon curing the aforementioned formulations was tested using a Discovery HR-2 rheometer equipped with a 20 mm parallel plate geometry and an Omnicure (series 2000) optics attachment as the light source. 40 μL of each sample were loaded onto the bottom plate and the top geometry was lowered to obtain a gap size of 100 μm. Measurement duration was set to 120 seconds with angular frequency of 2 Hz and a 1% strain in which the sample was preconditioned for 30 seconds and then light was initiated for 6 seconds at 22 mW/cm². The obtained data is presented in FIG. 5. As can be seen, the storage modulus, and accordingly the elastic modulus, of CPM is lower than that of CMR, indicating the improved elasticity imparted by the introduction of the PEG moieties to the pre-polymerized collagen.

### Solubility:

DLP bio-printing generally requires using an ink formulation which contains a dye that absorbs well in the range of 365 nm to 405 nm. This is a perquisite for printing fine structures with a 10-micrometer resolution. Most water soluble dyes that are usable in DLP contain a sulfate, or other negatively charged groups that impart water solubility.

Since collagen is a positively-charged protein, featuring a plurality of positively-charged groups, the presence of dyes that feature a sulfate, or other negatively charged groups, may lead to physical crosslinking or simply precipitation (in accordance with the Hofmeister series). This poses a severe limitation and enforces a use of a dye that is both water-soluble, biocompatible, non-toxic, features the desired absorption and does not feature negatively charged groups. Such dyes are not readily available, and when used, require a high amount for obtaining good resolution without precipitation.

Accordingly, as can be seen in FIG. 6, left vial, when a 10 mg/mL solution of CMR is mixed with 1 gram of UV386a, an exemplary polysulfate dye, precipitation is observed.

As can be further seen in FIG. 6, right vial, when a 10 mg/mL solution of CPM is mixed with 1 gram of UV386a, the solution remained clear, with no significant precipitation. Without being bound by any particular theory, this phenomenon may be explained by the high polarity of the PEG moieties, which enhances the solubility of the protein even in the presence of polysulfates, and/or by the interference of the PEG moieties with the physical cross-linking between the sulfate groups and the positively-charged groups of the collagen.

The optical density of the two tested solutions was measured at 600 nm. The O.D. of the CMR was 2.181 and of the CPM solution 0.294, nearly an order of magnitude.

This circumvents the imposed restriction in selecting suitable dyes and allows using negatively-charged dyes.

### Preparation of Methacrylated Collagen-Metacrylated PEG Conjugate

The present inventors have conceived also a “hybrid” conjugate in which the collagen-methacrylated PEG conjugate as described hereinabove is further modified by covalently attaching methacrylate groups directly to the collagen, as follows:

10 mL of CPM (3.0 mg/mL) prepared as described hereinabove were added into a 20-mL amber flask. 1.4 mL of 2M MOPS buffer solution pH=7.5 were then added followed by 400 microliters of 4M NaCl. The solution was cooled to 4° C. and 1.5 microliters of methacrylic anhydride were then added. The solution was allowed to stir at 4° C. overnight. TNBS assay showed a degree of modification of 42%.

### Example 3

The present inventor has conceived utilizing vitamin B12 as a dye for absorbing light in a DLP process.

Vitamin B12 (180 mg) or 4-nitrophenol (43 mg) was dissolved in 6 mL of mQ water, along with Lithium phenyl-2,4,6-trimethylbenzoylphosphinate (200 mg). 1 gram PEG (6000)-diacrylate was added to the solution, and then CMR as described herein was added into the solution to yield a final concentration of 5 mg/mL CMR and a total volume of 20 mL was achieved by adding 10 mM HCL. The resulting mixtures were each stirred until a homogenous solution was obtained, and were used to 3D print (using a DLP printer) a hydrogel featuring 50 μm channels along the Z axis and 150 μm pores in the XY plane. The DLP printing with both formulations was performed successfully.

The viscosity of the two formulations was evaluated and compared. Measurements were performed on Discovery HR-2 rheometer equipped with a 40 mm cone plate of 1°. 300 μL of sample were loaded on the bottom geometry and the top geometry was lowered to obtain a gap of 32 μm. The temperature was kept at 22° C. and the viscosity values were obtain from shear rates ranging from 0.01-1000 l/s (3 points per decade). The obtained data is shown in FIG. 7.

As can be seen, at a sheer rate of up to at least 10 l/sec, the Vitamin B12 formulation exhibited a much lower viscosity compared to the 4-nitrophenol formulation, rendering it more suitable for biopriting applications. This is added to the inherent advantage of using the naturally-occurring Vitamin B12 as a dye that is easily washed away from the printed object without the need to meet regulation requirements for hazardous materials.

### Example 4

To test the effect of the amount of a photoinitiator (PI) LAP, 3 different formulations were prepared with variance only in LAP concentrations,

A formulation containing 0.5% LAP was prepared as follows:
900 mg LAP were dissolved in 9 mL milli Q water, and one gram PCL (2000)-PEG (20K)-PCL-(2000) diacrylate, 0.1 gram PEG-DA 3400 and 900 mg of PEG-DA 700, were added thereto, 3.0 mL of 10 mM HCl were then added and the solution was stirred until a clear solution was obtained. 5.8 grams of a CMR (16.9 mg/mL) solution was then added to obtain a 5 mg/mL solution.

Formulations containing 0.75% and 0.9% LAP were similarly prepared, using respective amounts of LAP.

The viscosity of each formulation was measured as described hereinabove and the obtained data is presented in FIG. 8. As can be seen, the amount of the PI affects the formulation's viscosity, mainly at a shear rate of up to 10 l/sec, with lower viscosities provided at lower PI amount,

### Example 5

The present inventors have conceived using minocycline as a photoabsorber (e.g., a dye substance as described herein) in 3D-bioprinting, and have studied its performance in DLP 3D-bioprinting processes that employ collagen-containing curable formulations.

In an exemplary study, a curable formulation that comprises curable rhCollagen featuring a plurality of methacrylate groups, which is also referred to herein as CMR, an aqueous carrier, one or more additional curable polymeric materials (e.g., PEG-DA), and a photoinitiator, such as described in WO 2018/225076, was used.

In an exemplary procedure, minocycline [Apollo Scientific], was dissolved in mQ water, along with Lithium phenyl-2,4,6-trimethylbenzoylphosphinate (LAP, a photoinitiator). PEG (6000)-diacrylate was added to the solution, followed by the addition of CMR and diluted HCl. The resulting mixtures were each stirred until a homogenous solution was obtained, and were used to 3D print (using a DLP printer) a hydrogel featuring 200 μm channels along the Z axis and in the XY plane

The effect of the concentration of minocycline on the hardening rate and degree of the formulation was tested, by determining the G′ values of each formulation during the curing step.

Measurements were performed using a Discovery HR-2 rheometer equipped with a 20 mm parallel plate geometry and an Omnicure (series 2000) optics attachment as the light source. 25-90 μL of each sample were loaded onto the bottom plate and the top geometry was lowered to obtain a gap size of 50-250 μm, respective to the drop volume. Measurement's duration was set to 120 seconds with angular frequency of 2 Hz and a 1% strain in which the sample was preconditioned for 30 seconds and then light was initiated, using an external UV 365 nm light source, for 60 seconds at 50 mW/cm².

Formulations containing 0, 0.04, 0.08, 0.12, 0.16 and 0.2, % by weight minocycline were tested and the results are presented in FIG. 9.

As can be seen, all the formulations have reached the maximum crosslinking level, indicating that at the high light energy used, the minocycline does not affect the crosslinking of the formulation, also at a very low concentration of 0.08% by weight.

No precipitation was observed following the inclusion of minocycline, indicating its suitability as a photoabsorber collagen-containing curable formulations such as described herein.

### Example 6

A conjugate comprising collagen (rh-collagen as described herein) featuring a plurality of methacrylic groups attached to a portion of its lysine residues and a plurality of PEG moieties (methoxy-capped, non-curable) was synthesized as depicted in FIG. 10.

In brief, 209.3 grams MOPS buffer were dissolved in 200 mL mQ water. The solution was titrated to pH 7.5 by adding 42 mL 1ON NaOH and mQ water were added to a volume of 500 mL. The solution was filtered via 0.45 μm filter. 10 mL of IM HCl were added to 990 mL mQ water and the solution was stirred for 5 minutes. 70 mL of recombinant human Type I Collagen (Collplant Ltd.) (20.3 mg/mL) were poured into 420 mL of 10 mM HCl, the solution was stirred and passed to a 1-Liter reactor and was cooled to 6° C. 23 mL of 4M NaCl were added followed by 53 mL of MOPS 2M buffer pH 7.5, and the solution was titrated to pH 8.0 by incrementally adding 10N NaOH (total 5.8 mL). 5.14 grams of MeO-PEG 5000-PNC (2 mol equivalents relative to the lysine residues; see, FIG. 10) were dissolved in 50 mL mQ water and the solution was added to the reactor, and the reaction mixture was stirred at 6° C. for about 24 hours.

111 microliters of Methacrylic anhydride were then added and the reaction was stirred at 6° C. for about 24 hours. The reaction was then quenched by adding 115 mL HCl IM to obtain a solution with a pH of 2.5. The product was purified via dialysis which was carried out with ten 500 mL portions of 10 mM HCl, by adding 500 mL of 10 mM HCl and reducing the volume in the vessel to 500 mL, repeatedly.

The obtained formulation features a viscosity of about 100 centipoises, G′ of about 46,000 Pa, and Shear recovery of about 93%, all determined as described herein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-48. (canceled)

49. A conjugate comprising collagen and a plurality of elastic moieties covalently attached to the collagen, wherein at least a portion of the elastic moieties feature a curable group.

50. The conjugate of claim 49, wherein said curable group is at a terminus of each of the elastic moieties.

51. The conjugate of claim 49, wherein said curable group is a (meth)acrylic group.

52. The conjugate of claim 49, wherein at least a portion of said elastic moieties are poly(alkylene glycol) moieties.

53. The conjugate of claim 49, wherein at least a portion, or each, of said elastic moieties comprise a poly(alkylene glycol) moiety that features an acrylic or a (meth)acrylic group at its terminus.

54. The conjugate of claim 49, wherein at least a portion of said elastic moieties are covalently attached to lysine residues of the collagen.

55. The conjugate of claim 54, wherein from 1 to 20, or from 1 to 10, % of lysine residues in the collagen have the elastic moieties covalently attached thereto.

56. The conjugate of claim 54, wherein at least a portion of said elastic moieties is attached to said lysine residues via a carbamate bond.

57. The conjugate of claim 49, wherein said collagen features a plurality of photocurable groups.

58. The conjugate of claim 49, wherein the collagen is a plant-derived recombinant collagen.

59. The conjugate of claim 49, wherein the collagen is a plant-derived recombinant human Type I collagen.

60. The conjugate of claim 57, wherein the collagen is a plant-derived recombinant human Type I collagen that feature said plurality of photocurable groups.

61. A curable formulation comprising the conjugate of claim 49 and an aqueous carrier.

62. The curable formulation of claim 61, further comprising at least one additional curable material.

63. The curable formulation of claim 62, wherein said additional material is or comprises a poly(alkylene glycol) that features at least one (meth)acrylic group at its terminus.

64. The curable formulation of claim 61, wherein said curable group is a photocurable group and said agent is a photoinitiator.

65. The curable formulation of claim 64, further comprising a dye substance that is capable of absorbing light at a wavelength of from 300 nm to 800 nm, or of from 300 to 450 nm.

66. The curable formulation of claim 65, wherein said dye substance has a plurality of negatively-charged groups.

67. The curable formulation of claim 65, wherein said dye substance is selected from UV386a, Vitamin B12, a quinoline and minocycline.

68. A process of additive manufacturing a three-dimensional object featuring, in at least a portion thereof, a collagen-based material, the process comprising sequentially forming a plurality of layers in a configured pattern corresponding to a shape of the object,
wherein at least a portion of said layers are formed of a modeling material formulation that comprises the curable formulation of claim 61,
thereby manufacturing the three-dimensional object.

69. A three-dimensional biological object obtainable by the process of claim 68.

70. A method of repairing a damaged tissue, the method comprising administering the three-dimensional biological object of claim 69 to a subject in need thereof.

71. The three-dimensional biological object of claim 69, being an artificial tissue or organ.

72. A method of constructing an artificial organ or tissue in a subject in need thereof, the method comprising implanting the three-dimensional biological object of claim 69 to the subject.

73. A process of additive manufacturing a three-dimensional object featuring, in at least a portion thereof, a collagen-based material, the process comprising:
selecting an additive manufacturing technique;
mixing at least a collagen that features a plurality of photocurable groups, a photoinitiator, optionally an aqueous carrier, and optionally other curable and/or non-curable components, to thereby prepare a modeling material formulation (e.g., a bioink composition), wherein an amount of said photoinitiator is selected so as to provide a viscosity that is suitable for said additive manufacturing technique;
sequentially forming a plurality of layers in a configured pattern corresponding to a shape of the object,
wherein at least a portion of said layers are formed of said modeling material formulation that comprises said collagen that features a plurality of photocurable groups,
thereby manufacturing the three-dimensional object.

74. The process of claim 73, wherein the collagen is a human Type I collagen.

75. The process of claim 73, wherein the collagen is a plant-derived recombinant collagen.

76. The process of claim 73, wherein the collagen is a plant-derived recombinant human Type I collagen.

77. The process of claim 73, wherein said photoinitiator is an acyl phosphine oxide type photoinitiator.

78. A curable formulation comprising a collagen that features a plurality of photocurable groups covalently attached thereto, an additional curable material that features at least photocurable group, a photoinitiator and a dye substance that is capable of absorbing light at a wavelength of from 300 nm to 800 nm, or of from 300 to 450 nm.

79. The curable formulation of claim 78, wherein:
said collagen is a plant-derived recombinant human collagen; and/or
said additional curable material is or comprises a poly(alkylene glycol) moiety that features at least one acrylic or a (meth)acrylic group at its terminus; and/or
said photoinitiator is an acyl phosphine oxide type photoinitiator; and/or
said dye substance is or comprises minocycline; and/or
said dye substance is or comprises Vitamin B12.


SEQUENCE LISTING (16 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens [sequence omitted]

SEQ ID NO: 3 - protein, 1464 aa, Homo sapiens; identical to SEQ ID NO: 1

SEQ ID NO: 4 - protein, 1366 aa, Homo sapiens [sequence omitted]

SEQ ID NO: 5 - protein, 1489 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1389 aa, Homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 7 - DNA, 5927 bp, Homo sapiens
  source 1..5927: mol_type = genomic DNA
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 5411 bp, Homo sapiens
  source 1..5411: mol_type = genomic DNA
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 9 - DNA, 4467 bp, Homo sapiens
  source 1..4467: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggttccccaggagctgatggcccagctggtgctccaggaactccaggccctcaaggtattgctggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctc

SEQ ID NO: 10 - DNA, 4167 bp, Homo sapiens
  source 1..4167: mol_type = genomic DNA
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaag

SEQ ID NO: 11 - DNA, 1633 bp, synthetic construct
  misc_feature 1..1633: note = Synthetic sequence containing the coding regions of the vascularsignal sequence of barley gene for Thiol protease aleurainprecursor fused to the human Prolyl 4-hydroxylase beta subunitand flanking regions
  source 1..1633: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgagctc

SEQ ID NO: 12 - DNA, 1723 bp, synthetic construct
  misc_feature 1..1723: note = Synthetic sequence containing the coding regions of the vascularsignal sequence of barley gene for Thiol protease aleurainprecursor fused to the human Prolyl 4-hydroxylase alpha-1 subunitand flanking regions
  source 1..1723: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 13 - DNA, 2888 bp, synthetic construct
  misc_feature 1..2888: note = Synthetic sequence containing the coding regions of the vascularsignal sequence of barley gene for Thiol protease aleurainprecursor fused to the human Lysyl hydroxylase 3 and flankingregions
  source 1..2888: mol_type = other DNA
gcgaattcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccagatcttaaaccatggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccagaattcgcg

SEQ ID NO: 14 - protein, 45 aa, synthetic construct
  REGION 1..45: note = Vacuolar targeting sequence of the thiol protease aleurainprecursor (NCBI accession P05167GI:113603)
MAHARVLLLALAVLATAAVAVASSSSFADSNPIRPVTDRAASTLA

SEQ ID NO: 15 - protein, 1469 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 16 - DNA, 4416 bp, Homo sapiens
  source 1..4416: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggtggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctctaatgagct